(19) United States
(12) Patent Application Publication
(10) Pub. No.: US 2010/0028892 A1
(43) Pub. Date: Feb. 4, 2010

(54) YEAST BASED EXPRESSION OF PROTEASES AND METHODS OF USE

(75) Inventors: John C. Reed, Rancho Santa Fe, CA (US); Hideki Hayashi, Nagasaki (JP); Michael Cuddy, Carlsbad, CA (US)

Correspondence Address: DLA PIPER LLP (US), 4365 EXECUTIVE DRIVE, SUITE 1100, SAN DIEGO, CA, 92121-2133, US

(73) Assignee: Burnham Institute for Medical Research, La Jolla, CA (US)

(21) Appl. No.: 12/504,600

(22) Filed: Jul. 16, 2009

Related U.S. Application Data
(60) Provisional application No. 61/081,363, filed on Jul. 16, 2008.

(51) Int. Cl. C12Q 1/68

(52) U.S. Cl. 435/6

(57) ABSTRACT

This disclosure generally relates to components and methods of using a high throughput screening (HTS) systems for intracellular proteases, using Caspases as a prototype. Genetic systems are disclosed for monitoring exogenous caspase activation pathways in the yeast, Saccharomyces cerevisiae. The yeast-based cellular systems permit facile expression of proteases (e.g., caspase) and protease-activating proteins in combinations that reconstitute entire mammalian pathways in these simple eukaryotes. Among the assay methods integrated into the yeast system are cleavable reporter gene activators, in which protease-mediated cleavage activates a transcription factor. Exemplary systems rely, singly or in concert, on exogenous recombinant caspases and exogenous upstream activators of caspases to cleave a chimeric protein giving rise to a transcription factor which induces the expression of the LacZ and LEU2 genes. The activities of these genes result in colored cultures and impart the ability of the yeast to grow in leucine deficient media. The intensity of the color is measured by colorimetry and quantified with OD units. The OD units are directly proportional to the activity of the caspase in the system. The method of quantification is referred to as the “readout”.

### CROSS REFERENCE TO RELATED APPLICATION(S)

This application claims the benefit of priority under 35 U.S.C. § 119(e) of U.S. Ser. No. 61/081,363, filed Jul. 16, 2008, the entire content of which is incorporated herein by reference.

### GRANT INFORMATION

This invention was made in part with government support under Grant No. GM085255 awarded by the NIH. The United States government may have certain rights in this invention.

### BACKGROUND OF THE INVENTION

1. Field of the Invention

The disclosure generally relates to components and methods of using a high throughput screening (HTS) systems for intracellular proteases, using Caspases as a prototype. Disclosed are yeast-based cellular systems that permit facile expression of proteases and protease-activating proteins in combinations that reconstitute entire mammalian pathways in these simple eukaryotes. Among the assay methods integrated into the yeast system are cleavable reporter gene activators, in which protease-mediated cleavage activates a transcription factor.

2. Background Information

Endopeptidases (“proteases”) play critical roles in many biological processes and are often excellent targets for drug discovery. Development of high throughput screening (HTS) assays using purified proteases can be relatively straightforward or it can be quite challenging, particularly when multi-component systems are required to achieve protease activation. Also, due to similarity of the active sites of some groups of proteases, selectivity of chemical inhibitors can be difficult if not impossible to achieve, highlighting the need for alternative screening methods for identifying compounds that target upstream activators of proteases rather than directly inhibiting the protease of interest.

Caspases represent an excellent example of a family of intracellular endopeptidases for which novel HTS tools are desired. Caspases are cysteine proteases that are conserved throughout the animal kingdom. The human genome contains at least 10 genes encoding Caspases. These proteases often collaborate in complex proteolytic networks that encompass upstream initiators and downstream effectors, where upstream members of the Caspase family cleave and activate downstream members. Upstream initiator Caspases become activated through protein interactions involving assembly of multi-protein complexes that are difficult to reconstitute in vitro. The substrates cleaved by active Caspases are responsible for either apoptotic cell death or for cytokine-mediated inflammation, thus making these proteases attractive targets for drug discovery for a wide variety of degenerative diseases, ischemic disease, autoimmunity, inflammatory conditions, and some host-pathogen interactions.

Currently, there is no assay technology for high-throughput screening which reconstitutes the use and economy as those facilitated by the present disclosure.

### SUMMARY OF THE INVENTION

Herein are disclosed genetic systems for monitoring exogenous caspase activation pathways in the yeast, Saccharomyces cerevisiae. These systems rely, singly or in concert, on exogenous recombinant caspases and exogenous upstream activators of caspases to cleave a chimeric protein giving rise to a transcription factor which induces the expression of the LacZ and LEU2 genes. The activities of these genes result in blue cultures and impart the ability of the yeast to grow in leucine deficient media. The blue color is due to the cleavage of X-gal by the LacZ gene product, β-galactosidase. The intensity of the blue color is measured by colorimetry and quantified with OD units. The OD units are directly proportional to the activity of the caspase in the system. The method of quantification is referred to as the “readout”.

In a “Single Component” system an exogenous caspase is stably over-expressed and autoactivated in a yeast strain containing the transmembrane receptor, CD95/Fas, (“chimera”). The chimera is expressed such that most of its cytosolic domain is replaced by a chimeric transcription factor comprised of the DNA-binding domain of LexA and the transactivation domain of B42. Between Fas and the chimeric transcription factor (LexA-B42) domains are various numbers of tetrapeptide sequences known to be recognized and cleaved by various members of the Caspase family. Each genetic system of this disclosure contains an appropriately matched set of the appropriate caspase to the appropriate chimera. The activity of the entire system is monitored by the readout.

An embodiment of this disclosure provides for a method using a single-component system exemplified above for drug screening assays wherein the activities of Caspase 1, 2, 3, 4, 5, 6, 7, and 9 are measured and compounds that inhibit Caspase 1, 2, 3, 4, 5, 6, 7, and 9 are identified.

Furthermore, an embodiment of this disclosure provides for a method using a single-component system exemplified above for drug screening assays which may utilize any other substrate of the LacZ gene, beta-galactosidase, for quantification.

In a “Two-Component” system an exogenous caspase is expressed at a low level to maintain its inactive pro-caspase form along with its exogenous cogent upstream activator. The level of expression is engineered to prevent autoactivation such that the interaction of the upstream activator with the pro-caspase results in the activated caspase and the readout. The level of expression is engineered to impact assay performance by manipulating several variables of vector construction, including promoter strength, promoter inducibility, vector copy number, and number of operators (transcription factor binding sites) in a reporter gene.

An embodiment of this disclosure provides for a method using a two-component system exemplified above for drug screening assays where the activities of ASC, RAIDD, FADD, Apaf, and active caspase-9 are measured and compounds that inhibit the interaction or activity of ASC with pro-caspase-1, RAIDD with pro-caspase-2, FADD with pro-caspase-8 or pro-caspase-10, an active mutant of Apaf with pro-caspase-9, active caspase-9 with pro-caspase-3 or procaspase-7 are identified.

An embodiment of this disclosure provides for a method using a two-component system exemplified above for drug screening assays where the activities are used to identify or discover other or novel upstream activators of pro-caspase-1, pro-caspase-2, pro-caspase-3, pro-caspase-4, pro-caspase-5, pro-caspase-6, pro-caspase-7, pro-caspase-8, pro-caspase-9, and pro-caspase-10 via the introduction of a cDNA library or other expression conveyance to screen for biomolecular activators of the pro-caspases.

Furthermore, an embodiment this disclosure provides for a method using a two-component system exemplified above for drug screening assays which may utilize any other substrate of the LacZ gene, beta-galactosidase, for quantification

In a “Plural-Component” system an exogenous caspase is expressed in its inactive pro-caspase form along with its exogenous cogent activation pathway components consisting of two or more additional upstream proteins required for activation of the caspase and the subsequent readout. The levels of expression of the components are engineered to prevent caspase activation unless all of the components are present.

An embodiment of this disclosure provides for a method using a plural-component system exemplified above for drug screening assays where the activities of FAS, FADD, DR5, ASC, or NALP1 are measured and compounds that inhibit the interaction or activity of FAS with FADD, DR5 with FADD, FADD with pro-caspase-8 or pro-caspase-10, ASC with NALP1, or NALP1 with pro-caspase-1.

An embodiment of this disclosure provides for a method using a plural-component system exemplified above for drug screening assays where the activities are used to identify or discover other or novel upstream activators of FADD and NALP1 via the introduction of a cDNA library or other expression conveyance to screen for biomolecular activators of FADD or NALP1.

Furthermore, an embodiment this disclosure provides for a method using a plural-component system exemplified above for drug screening assays which may utilize any other substrate of the LacZ gene, beta-galactosidase, for quantification.

Another embodiment of the present disclosure incorporates the use of two-component and/or plural-component systems and by extension their specific examples to identify caspase activation protease networks in yeast, and to isolate and study an exogenous protease network in a cellular context.

An embodiment disclosed includes methods of performing chemical library screens to validate the performance of the assays and to further identify caspase activation protease networks in yeast, and to isolate and study an exogenous protease network in a cellular context. The screens are conducted such that all assay components may be added in automated fashion using integrated robotic liquid handling systems, moving the plates initially into carousels that hold ˜180 plates at room temperature, and then manually applying seals (breathable sealing film from Axygen Scientific) to reduce evaporation, and moving the bar-coded plates to a 30° C. incubator for the required time, generally 2-4 days. Each assay plate will contain a row of positive (min) and a row of negative (max) controls that do not receive compounds but that receive DMSO in volumes equivalent to the amount of DMSO in which compounds will be supplied. At the conclusion of the 72 hrs incubation, plates are robotically delivered to one of the integrated multi-purpose plate readers for reading at OD620 nm. Programmable robotic workstations sequence the additions of reagents, minimizing variations in incubation times. Data from primary screens are uploaded directly from plate readers into computers with customized Microsoftexcel software, set up to calculate Z′ factor for each plate, and with hit determinations set at 50% of the mean value for the negative control values.

An embodiment disclosed utilizes library screens which may be performed at several different concentrations (eg., 20, 10, and 5 μM) to compare the hit rates, and to empirically determine an acceptable concentration for conducting large-scale library screens. It is often best to employ as high a concentration of compounds as possible to maximize the chance of identifying active compounds (avoiding false-negatives), but balance that against an excessive frequency of hits (avoiding false-positives). A general rule of thumb is to empirically adjust compound screening concentrations to achieve a hit rate of 0.1-0.5%. Thus, before undertaking starting a large library screen, pilot experiments may be utilized to optimize results such as empirically determining the effects of DMSO on assay performance, through pilot experiments where increasing concentrations of DMSO (from 1-10% volume) are added to the positive and negative controls and the impact on assay signal and stability is determined. Second, when progressing with larger libraries (e.g., 50K Chembridge), it is useful to determine the stability of the positive and negative controls from plate to plate, assessing the assay performance as time is extended from minutes to hours, and calculating Z′ for each plate as the quality of the assay's performance is assessed in true screening mode.

For HTS assays we have measured β-galactosidase produced by yeast carrying the caspase-cleavable reporter proteins, and assayed the calorimetric product (OD620 nm) derived from X-gal substrate in 384 well plates, as a measure of the lac Z reporter gene activity.

An embodiment of the present disclosure comprises a high throughput screening of lac-Z reporter gene activity by measuring β-galactosidase produced by yeast carrying caspase-cleavable reporter proteins; and assaying the calorimetric product derived from x-gal substrate in 384 well plates, at an optical density of 620 nm.

### BRIEF DESCRIPTION OF THE DRAWINGS

FIG. 1 Illustrates the Typical Prodomain Size of Upstream and Downstream Caspases.

FIG. 2 Consisting of FIG. 2A and FIG. 2B is a schematic representation of an embodiment of the present disclosure using a single-component system as a genetic system for monitoring caspase 1 activity in yeast cells.

FIG. 3 Demonstrates the resulting substrate specifities of the caspases expressed in yeast when using a single-component system.

FIG. 4 Consisting of FIG. 4A and FIG. 4B is a schematic representation of an embodiment of the present disclosure using a two-component system for caspasel activators in yeast cells.

FIG. 5 Consisting of FIG. 5A, FIG. 5B, FIG. 5C, FIG. 5D, FIG. 5E, FIG. 5F and FIG. 5G demonstrates the resulting caspase activation in yeast when using a two-component system.

FIG. 6 Consisting of FIG. 6A, FIG. 6B, FIG. 6C and FIG. 6D) is a schematic representation of an embodiment of the present disclosure using a plural-component system for caspase-8 activation in yeast cells.

FIG. 7 Consisting of FIG. 7A, FIG. 7B, FIG. 7C, FIG. 7D, FIG. 7E, FIG. 7F and FIG. 7G demonstrates the resulting caspase-8 and caspase-10 activation in yeast when using a plural-component system.

FIG. 8 Consisting of FIG. 8A, FIG. 8B and FIG. 8C demonstrates the basis for the cleavable reporter assay when using of a two-component and/or plural component system when screening for NALP1.

FIG. 9 Consisting of FIG. 9A, FIG. 9B and FIG. 9C demonstrates the basis for the cleavable reporter assay when using of a two-component and/or plural component system when screening for NLRs.

FIG. 10 Demonstrates the optimization of signal:noise ratio in microtiter plates when performing high throughput screening of cDNA libraries with the two or plural-component systems.

FIG. 11 Consisting of FIG. 11A, FIG. 11B and FIG. 11C demonstrates the validation of single, two, and plural-component yeast-based Caspase assays using pharmacological inhibitor of Caspases.

FIG. 12 Consisting of FIG. 12A, FIG. 12B and FIG. 12C demonstrates HTS Implementation of yeast-based NALP1 inflammasome assay. (A) NALP1 yeast screen controls, and a Yeast Screen of a LOPAC compound library for NALP1.

FIG. 13 Consisting of FIG. 13A, FIG. 13B and FIG. 13C demonstrates the resulting substrate sequence- and Caspase activity-dependent cleavage of S1, S2, and S9.

FIG. 14 Demonstrates a Flow chart for cDNA library screening using a reporter gene strategy based on cleavable transcription factor using a S1 substrate.

FIG. 15 Demonstrates a Flow chart for cDNA library screening using a reporter gene strategy based on cleavable transcription factor using a S3/S7 substrate.

FIG. 16 Consisting of FIG. 16A and FIG. 16B demonstrates the resulting substrate specifities of the caspases expressed in yeast when using a single-component system.

FIG. 17 Consisting of FIG. 17A and FIG. 17B demonstrates the resulting Validation of yeast-based assays for effector Caspase activators—two and three-component systems.

FIG. 18 Consisting of FIG. 18A, FIG. 18B, FIG. 18C and FIG. 18D demonstrates the resulting validation of a yeast-based assays for effector Caspase activators when using a two component systems.

FIG. 19 Demonstrates the resulting specificity of upstream activators of initiator Caspases when using a 2-component system.

FIG. 20 Consisting of FIG. 20A and FIG. 20B demonstrates the resulting validation of a 3-component yeast-based Caspase assay reconstituting DISC.

FIG. 21 Demonstrates a Flow chart for cDNA library screening using 3 component system -application to death receptor cloning—Example Screening Strategy #1.

FIG. 22 Demonstrates a Flow chart for cDNA library screening using 3 component system—application to death receptor cloning—Example Screening Strategy #2.

FIG. 23 Demonstrates a Flow chart for cDNA library screening using 3 component system—application to death receptor cloning—Example Screening Strategy #3.

FIG. 24 Consisting of FIG. 24A and FIG. 24B demonstrates a schematic representation of a 3-component system used for cloning adapter protein that links Fas to pro-Caspase-10.

FIG. 25 Consisting of FIG. 25A and FIG. 25B demonstrates the resulting validation of adapter protein cloning system for Fas/FADD/Caspase-8/10: Reconstituted DISC.

FIG. 26 Demonstrates a Flow chart for cDNA library screening using 3-component system to clone adapters.

FIG. 27 Consisting of FIG. 27A, FIG. 27B and FIG. 27C demonstrates the resulting validation the cDNA cloning results in which the clones that activated the reporter genes were characterized by recovery of cDNA library plasmids and retransformation into yeast expressing Fas and pro-Caspase-10.

FIG. 28 Consisting of FIG. 28A, FIG. 28B and FIG. 28C graphically depicts the optimization of signal:noise ratio in microtiter plates using cell density.

FIG. 29 Consisting of FIG. 29A and FIG. 29B graphically depicts the optimization of signal:noise ratio in microtiter plates using time and X-gal concentration.

FIG. 30 Consisting of FIG. 30A, FIG. 30B, FIG. 30C and FIG. 30D demonstrates a diagram of plasmids for co-expression of Caspases and substrate cleavable transcription factors in yeast.

FIG. 31 Consisting of FIG. 31A, FIG. 31B, FIG. 31C and FIG. 31D demonstrates a diagram of plasmids for expression of upstream activators of Caspases and lacZ reporter gene in yeast.

FIG. 32 Consisting of FIG. 32A, FIG. 32B and FIG. 32C demonstrates a diagram of plasmids for functional screening of cDNA libraries and expression of upstream activators of Caspases.

### DETAILED DESCRIPTION OF THE INVENTION

Proteolytic processing of proteins is an irreversible post-translational modification of importance for a wide-variety of biological processes, including cell division, cell death, cell differentiation, innate immunity, host-pathogen interactions, and intracellular protein sorting and trafficking (Lopez-Otin C, and Overall CM. Protease degradomics: a new challenge for proteomics. Nat Rev Mol Cell Biol 2002; 3(7): 509-19). Consequently, proteases have emerged as promising targets for drug discovery for a wide variety of human diseases, including cancer, neurodegeneration, ischemic diseases, inflammation, and infectious diseases.

Proteases can be found in either intracellular or extracellular (or cell surface) locations, where they encounter their specific substrates. Among the intracellular families of proteases are Caspases, Calpains, Deubiquitinating enzymes and their homologs, and Separase. Still other proteases may have dual intracellular and extracellular lives. For example, Cathepsins are normally stored in lysosomes undergoing vesicular recycling between extracellular and intracellular compartments, but also are released into the cytosol under various pathological circumstances, promoting cell death (Cirman T, et al. Selective disruption of lysosomes in HeLa cells triggers apoptosis mediated by cleavage of Bid by multiple papain-like lysosomal cathepsins. J Biol Chem 2004; 279: 3578-87).

From the standpoint of high-throughput screening (HTS) for identifying chemical inhibitors of proteases, many endopeptidases (or at least their catalytic domains) can be readily produced in large quantities by recombinant DNA technology, purified, and formatted for HTS using convenient fluorigenic or colorimetric substrates. However, such assay formats have several limitations. First, standard HTS configurations for proteases are typically amenable only to screens for inhibitors, not activators of proteases. Second, assays requiring large amounts of purified, active protease can be hampered by difficulties in producing by recombinant methods or purifying from endogenous sources sufficient amounts of material, as well as by instability problems where purified proteases lose activity in vitro. Third, because one is often limited to using only the catalytic domain due to difficulties of expressing and purifying intact full-length proteases, opportunities to discover allosteric modulators of proteases are limited, with most of the standard assays limited to identification of compounds that target the active site in a competitive fashion. Fourth, when using single target systems consisting of purified protease or catalytic domain of protease, opportunities for identification of compounds that target other proteins involved in protease activation are lost. Fifth, several classes of intracellular endopeptidases exists as large families of closely related enzymes with structurally very similar active sites, making it difficult to achieve selective inhibitors in the absence of more context-dependent, multi-component systems that would provide the opportunity for selective inhibition. Sixth, some proteases are not active in purified form, requiring necessary cofactors (e.g. Separins involved in chromosome segregation and cell division) or requiring membranes (e.g. Presenilins (such as γ-secretase) involved in Amyloid-beta-peptide processing).

### Caspases

Caspases are intracellular cysteine proteinases with specificity for aspartic acid residues in the P₁ position of substrates. These proteases are well conserved throughout animal evolution, where they play essential roles in programmed cell death (apoptosis). In Caenorhabiditis elegans, for example, all programmed cell deaths that occur during the development of this simple animal depend upon the presence of an intact CED-3 gene, which encodes a Caspase (Yuan J, et al., The C. elegans cell death gene ced-3 encodes a protein similar to mammalian interleukin-1 beta-converting enzyme. CELL 1993; 75: 641-52). In humans, 12 genes encoding Caspases or Caspase-like proteins have been identified, 10 of which have been clearly and convincingly shown to possess protease activity and all showing absolute dependence on aspartic acid at the P1 position of substrates, including Caspases-1-10. Another member of the family, Caspase-12, is produced only in ˜1% of African populations, due to a polymorphism that produce a nonsense mutation that truncates the protein in most humans, and its proteolytic activity is questionable (Fischer H, et al., Human caspase-12 has acquired deleterious mutations. Biochem Biophys Res Comm 2002; 293: 722-6; and Saleh M, et al., Enhanced bacterial clearance and sepsis resistance in caspase-12-deficient mice. Nature 2006; 440: 1064-8). Protease activity for the remaining member, Caspase-14, has not been demonstrated to date (Kuechle M K, et al., Caspase-14, a keratinocyte specific caspase: mRNA splice variants and expression pattern in embryonic and adult mouse. Cell Death Differ 2001; 8: 868-70). In addition to apoptosis, other biological roles for Caspases have been elucidated, the most studied of which is inflammation. Of the 10 established human Caspases, 3 of them cleave and activate pro-inflammatory cytokines (particularly pro-IL-1b, pro-IL-18, pro-IL-32), having major roles in host-defense and inflammation (Reed J C. Caspases and cytokines: roles in inflammation and autoimmunity. Adv Immunol 1999; 73 :265-99).

Caspases exist as inactive zymogens in all animal cells but can be activated by proteolytic cleavage of their proforms at conserved aspartic acid residues, thus generating the subunits of the enzymatically active proteases which consist of heterotetramers comprised of two large and two small subunits. Because caspases both cleave substrates at Asp residues and are themselves activated by cleavage at Asp residues, the potential for proteolytic cascades exists and indeed has been documented (reviewed in (Salvesen G S, et al., Intracellular signaling by proteolysis. Cell 1997; 91 :443-6; and Thornberry N A, and Lazebnik Y. Caspases: enemies within. Science 1998; 281: 1312-6). The concept of upstream initiator and downstream effector caspases that operate within a proteolytic cascade thus has emerged (Salvesen G S, et al., Cell 1997; 91: 443-6; and Alnemri E S, et al. Human ICE/CED-3 Protease Nomenclature. Cell 1996; 87: 171).

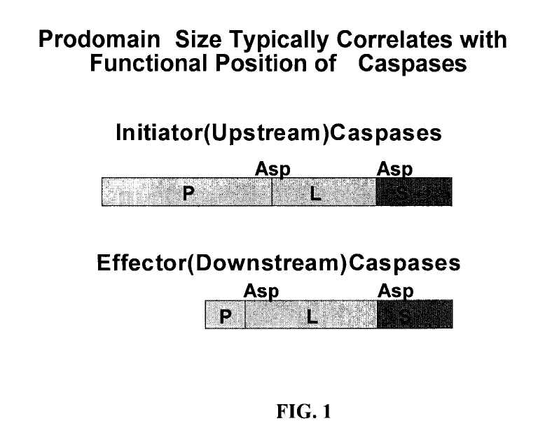

FIG. 1 helps illustrate that relevant to their mechanisms of activation, all of the upstream initiator Caspases contain large prodomains and many of these prodomains have been shown to bind other proteins involved in triggering the cascade. The downstream Caspases, which function as the ultimate effectors of apoptosis, uniformly possess small prodomains and are probably activated predominantly if not exclusively by proteolytic cleavage by upstream Caspases. The irreversible cleavage of specific protein substrates in cells by these downstream effector Caspases is what directly or indirectly then accounts for the biochemical and morphological changes that are recognized as apoptosis. Among the 10 established human Caspases, only 3 have small prodomains and are considered downstream executioners (Caspases-3, 6, 7). The other 7 members of the family have large N-terminal prodomains containing signature domains involved in protein-protein interactions that provide the basis for initiating the proteolytic pathway.

### Substrate Specificity of Caspases

The 3D-structure of the active sites of most human Caspases accommodates tetrapeptide substrates, in which Aspartic acid at the P1 position is invariant. In Drosophila, Glutamic acid can also be tolerated for some of the Caspases (Hawkins C J, et al., The drosophila caspase DRONC cleaves following glutamate or aspartate and is regulated by DIAP1, HID; and GRIM. J Biol Chem 2000; 275: 27084-93). Differences in the geometry and side-chains of amino acids surrounding the active site dictate preferences among Caspases for different tetrapeptide substrates. Surveys of peptide libraries have elaborated activity profiles for most of the human Caspases (Talanian R, et al. Substrate specificities of caspase family proteases. J Biol Chem 1997; 272: 9677-82), as summarized in Table 1. These peptidyl substrate preferences match the known cleavage sites of endogenous proteins that are hydrolyzed by specific Caspases. For example, the WEHD sequence preferred by the inflammatory Caspase is found in pro-IL-1b and pro-IL-18, while the DEVD sequence preferred by downstream apoptotic proteases Caspases-3 and -7 is found in enzymes responsible for the characteristic chromatin condensation and DNA fragmentation associated commonly with apoptosis (Timmer J C, and Salvesen G S. Caspase substrates. Cell Death Differ 2007; 14(1): 66-72).

TABLE 1

| Caspase | Substrate | Cleavage site | Effect |
|---|---|---|---|
| Caspase 3 and 7 | Consensus | DEVD/G | |
| | ICAD | DETD/S | Initiates DNA fragmentation |
| | PARP | DEVD/G | Inactivates DNA repair |
| | PKCδ | DMQD/N | Activates kinase |
| | MEKK-1 | DTVD/G | Activates kinase |
| | U1-70kD | DPGD/G | Inactivates mRNA splicing |
| Caspase 6 | Consensus | VEID/G | |
| | Lamin A | VEID/N | Nuclear collapse |
| | Keratin 18 | VEVD/A | Cytoskeletal collapse |
| Caspase 8 | Consensus | IETD/G | |
| | procaspase 3 | IETD/S | Activates caspase |
| | procaspase 7 | IQAD/S | Activates caspase |
| | BID | LQTD/G | Cytochrome c release |
| Caspase 1 | Consensus | WEHD/G | |
| | proIL-1β | YVHD/A | Activates IL-1β |
| | proIGIF (IL-18) | LESD/N | Activates IGIF |

### Mechanisms of Activating Upstream Initiator Caspases

One of the major pathways for Caspase activation involves the TNF family of cytokine receptors (reviewed in Wallach D, et al., Cell death induction by receptors of the TNF family: towards a molecular understanding. FEBS Lett 1997; 410: 96-106, and Ashkenazi A, and Dixit V M. Death receptors: signaling and modulation. Science 1998; 281: 1305-8). Several TNF family receptors are known to transduce signals that result in apoptosis, including TNF-R1(CD120a), Fas (CD95), DR3 (Wsl-1; Tramp); DR4 (Trail-R1); DR5 (Trail-R2); and CAR-1. These death receptors contain a conserved cytosolic domain known as a Death Domain (DD) that is responsible for recruiting adapter proteins such as Fadd/Mort-1 to the receptor complex after binding of ligand. The Fadd/Mort-1 protein contains both a DD domain and an additional protein-interaction domain called a Death Effector Domain (DED). The DED of Fadd/Mort-1 binds certain caspases which contain homologous DEDs within their prodomains, caspases-8 and -10. The oligomerization of caspases within the death receptor complex results in trans-processing of the zymogens (Muzio M, et al., An induced proximity model for caspase-8 activation. J Biol Chem 1998; 273: 2926-30; and Yang X, et al., Autoproteolytic activation of pro-caspases by oligomerization. Mol Cell 1998; 1: 319-25), which contain low levels of proteolytic activity even before undergoing processing to the fully active protease. Processing of caspase-8 removes the DED-containing prodomain, thus releasing the activated protease into the cytosol where it can cleave and activate other downstream pro-caspases such as caspase-3 (Stennicke H R, et al. Pro-caspase-3 is a major physiologic target of caspase-8. J Biol Chem 1998; 273: 27084-90).

The pathway for apoptosis induction employed by the TNF family death receptors is sometimes referred to as the “Extrinsic Pathway.” This pathway for caspase activation plays a critical role in the mechanisms used by immune cells to induce apoptosis in target cells. Cytolytic T-cells, for example, employ death ligands, particularly Fas-Ligand (Fas-L), as a weapon for inducing apoptosis in target cells (Nagata S, and Golstein P. The Fas death factor. Science 1995; 267(5203): 1449-56). Several anti-apoptotic proteins that contain Death Effector Domains have also been described that operate as suppressers of apoptosis induced by TNF-family death receptors. These proteins contain DEDs, which allow them to interact with other DED-proteins, such as Fadd, pro-caspase-8, and pro-caspase-10, thereby interfering with assembly of the multiprotein complexes required for death receptor-mediated activation of caspases (Irmler M, et al. Inhibition of death receptor signals by cellular FLIP. Nature 1997; 388: 190-5; and Tschopp J, et al., Inhibition of Fas death signals by FLIPs. Curr Opin Immunol 1998; 10: 552-8). For example, the cellular protein Flip (reviewed in Wallach D. Placing death under control. Nature 1997; 388: 123-6) is such an anti-apoptotic protein, representing a homologue of caspases-8 and -10 that contains DED domains but lacks proteolytic activity.

Standing apart from the Death Receptors is another mechanism for achieving initiator Caspase activation that relies on nucleotide-binding oligomerization domains in Caspase-binding proteins to assemble multi-protein complexes, sometimes referred to as “apoptosome” or “inflammasomes”, depending on whether the Caspases activated are involved in cell death versus inflammation. The prototype for this Caspase activation mechanism comes from pioneering work in Caenorhabiditis elegans, where the only documented mechanism of inducing caspase activation depends on CED-4. The CED-4 protein is an ATP-binding protein and putative ATPase, which binds to pro-CED-3, an initiator caspase (Seshagiri S, and Miller L K. Caenorhabditis elegans CED4 stimulates CED-3 processing and CED-3-induced apoptosis. Curr Biol 1997; 7: 455-60; and Chinnaiyan A, et al., Role of CED-4 in the activation of CED-3. Nature 1997; 388: 728-9). Binding of ATP to CED4 induces conformational changes that result in oligomerization of CED-4 proteins, followed by binding to pro-CED-3 (Yang X, et al., Essential role of CED-4 oligomerization in CED-3 activation and apoptosis. Science 1998; 281: 1355-7). The oligomerization of associated pro-CED-3 proteins is thought to bring these zymogens into close proximity, allowing them to dimerize and become active, typically cleaving themselves or trans-processing each other, thereby generating the fully active autonomous proteases. The first mammalian homologue of CED-4 identified was Apaf-1 (Apoptotic Protease Activating Factor-1). The human Apaf-1 protein shares in common with CED-4 the presence of a nucleotide binding NB-ARC domain and a caspase-binding CARD (CAspase Recruitment Domain) domain. However, Apaf-1 is structurally more complex than the worm CED-4 (Zou H, et al., Apaf-1, a human protein homologous to C. elegans CED-4, participates in cytochrome c-dependent activation of caspase-3. Cell 1997; 90: 405-13), in that it also contains 12-14 copies of a W) domain at its C-terminus. The WD domains function as negative-regulatory region, rendering Apaf-1 inactive until bound by cytochrome c from mitochondria (Li P, el al. Cytochrome c and dATP-dependent formation of Apaf-1/Caspase-9 complex initiates an apoptotic protease cascade. Cell 1997; 91: 479-89). The presence of this negative-regulatory domain thus defines a fundamental difference between the human Apaf-1 and worm CED-4 proteins. The human protein requires an activation step to interact with caspases and induce cell death, whereas the worm CED-4 protein has constitutive caspase-binding and death-inducing activity (reviewed in Reed J C. Cytochrome C: Can't live with it; Can't live without it. Cell 1997; 91 :559-62). The only known mechanism for activating Apaf-1 is cytochrome c, which binds to Apaf-1 apparently relieving the repression applied by the WD domains (Li P, et al. Cell 1997; 91: 479-89). Cytochrome c is normally sequestered inside mitochondria, between the inner and outer membranes of these organelles. However, it becomes released into the cytosol following exposure of cells to a variety of pro-apoptotic stimuli, including chemotherapeutic drugs (Liu X, et al., Induction of apoptotic program in cell-free extracts: requirement for dATP and Cytochrome C. Cell 1996; 86: 147-57; Kluck R M, et al., The release of cytochrome c from mitochondria: a primary site for Bcl-2 regulation of apoptosis. Science 1997; 275 :1132-6; and Yang J, et al., Prevention of apoptosis by Bcl-2: release of cytochrome c from mitochondria blocked. Science 1997; 275 :1129-32). The apical caspase in the cytochrome c/mitochondrial pathway is pro-caspase-9. Pro-caspase-9 possesses an N-terminal CARD domain that allows it to bind the CARD domain of Apaf-1. Oligomerization of Apaf-1 induced by cytochrome c and ATP (or dATP) brings associated pro-caspase-9 zymogens into close proximity, allowing them to dimerize and become active (Zou H, et al., An APAF-1 cytochrome c multimeric complex is a functional apoptosome that activates procaspase-9. J Biol Chem 1999; 274: 11549-56; and Saleh A, et al., Cytochrome c and dATP-mediated oligomerization of Apaf-1 is a prerequisite for procaspase-9 activation. J Biol Chem 1999; 274: 17941-5). The caspase-activation pathway mediated by Apaf-1 as a result of cytochrome c release from mitochondria is sometimes referred to as the “Intrinsic Pathway.” The complex of Apaf1/cytochrome c/Caspase-9 is known as the “apoptosome” a donut-like structure with two stacked rings, each ring having stoichiometry of 7:14:7, based on high resolution cryo-electron microscopy imaging studies and other methods (Salvesen G S, and Renatus M. Apoptosome: The seven-spoked death machine. Develop Cell 2002; 2: 256-7).

An analogous system for achieving initiator Caspase activation has been described for inflammatory caspases, in which members of the NLR family (“NACHT+LRR” proteins, also known as “NOD-Like Receptors”) oligomerize to form a platform for recruitment and activation of inflammatory caspases such as Caspase-1, 4, and 5 in humans (reviewed in Martinon F, et al., The Inflammasome: A molecular platform triggering activation of inflammatory caspases and processing of proIL-b. Mol Cell 2002; 10: 417-26). NLRs consist of a central nucleotide-binding domain called NACHT, flanked on the C-terminus by Leucine Rich Repeats (LRRs) and on the N-terminus by either CARDs or PYRIN Domains (PYDs). The LRRs of NLRs keep these proteins in an inactive monomeric state, until bound by pathogen-derived products such as components of bacterial peptidoglycan. In vitro reconstitution studies by our laboratory have shown that upon binding pathogen-derive ligands, a conformation change occurs in NLRs that renders the NACHT domain competent to bind nucleotide triphosphates (NTPs) and undergo NACHT-dependent oligomerization (Faustin B, et al. Reconstituted NALP1 inflammasome reveals two-step mechanism of Caspase-1 activation. Molecular Cell 2007; 25(5): 713-24). The N-terminal CARD and PYD domains of NLRs bind directly to the CARD of inflammatory caspases or indirectly link to them via the bipartite adapter protein ASC, which contains both a CARD and PYD (Stehlik C, and Reed J C. The PYRIN connection: novel players in innate immunity and inflammation. J Exp Med 2004; 200: 551-8). The complex of NLR/ASC/Caspase-1 is called the “inflammasome” (Martinon F, and Tschopp J. NLRs join TLRs as innate sensors of pathogens. Trends Immunol 2005; 26: 447-54). Cryo-EM imaging studies suggest that the NALP1 (NLRP1) inflammasome is a donut-like structure, akin to the apoptosome (Faustin B, et al. Molecular Cell 2007; 25(5): 713-24).

Other mechanisms of mammalian Caspase activation have also been described, including the PIDDosome, a multiprotein complex involving the p53-inducible protein PIDD, the adapter protein RAIDD, and Caspase-2 (Tinel A, and Tschopp J. The PIDDosome, a protein complex implicated in activation of caspase-2 in response to genotoxic stress. Science 2004; 304: 843-6). PIDD contains an oligomerization domain and Death Domain (DD). The DD of PIDD binds a complementary DD found in the bipartite adapter protein RAIDD, which contains a DD and CARD. The CARD of RAIDD binds the CARD of pro-Caspase-2, thus providing the necessary link to the protease.

### Phenotypes of Animal Caspases Expressed in Yeast

The phenotypes of animal Caspases expressed in yeast have been explored. (Kamada S, et al. A cloning method for caspase substrates that uses the yeast two-hybrid system: Cloning of the antiapoptotic gene gelsolin. Proc Natl Acad Sci USA 1998; 95: 8532-7; Hawkins C J, et al., A cloning method to identify caspases and their regulators in yeast: Identification of Drosophila DIAP1 as an inhibitor of the Drosophila caspase DCP-1. Proc Natl Acad Sci USA 1999; 96: 2885-90; Kang J, et al. Cascades of mammalian caspase activation in the yeast Saccharomyces cerevisiae. J Biol Chem 1999; 274: 3189-98; Zhang H, and Reed J C. Studies of apoptosis proteins in yeast. In: Schwartz L, Ashwell, editors. Methods in Cell Biology. 2nd ed: Academic Press; 2001. p. 453-68; and Jin C, and Reed J C. Yeast and apoptosis. Nature Rev Mol Cell Biol 2002; 3: 453-9). Several lessons have been learned from these studies. First, over-expression of some zymogen forms of Caspases in yeast is sufficient to result in their activation, particularly the initiator Caspases, where it is likely that their CARD or DED-containing prodomains mediate dimerization when proteins are over-expressed. Second, some animal Caspases kill yeast, whereas others do not. Third, for those non-lethal Caspases, it is possible to devise cleavable reporter systems, particularly transcription factors that activate reporter genes only when cleaved by Caspases (Hawkins C J, et al., Proc Natl Acad Sci USA 1999; 96: 2885-90).

The following examples are intended to illustrate but not limit the invention.

### EXAMPLE 1

### Title

### Development and Testing of a Cleavable Reporter Gene System for Assaying Protease Activity in Living Yeast

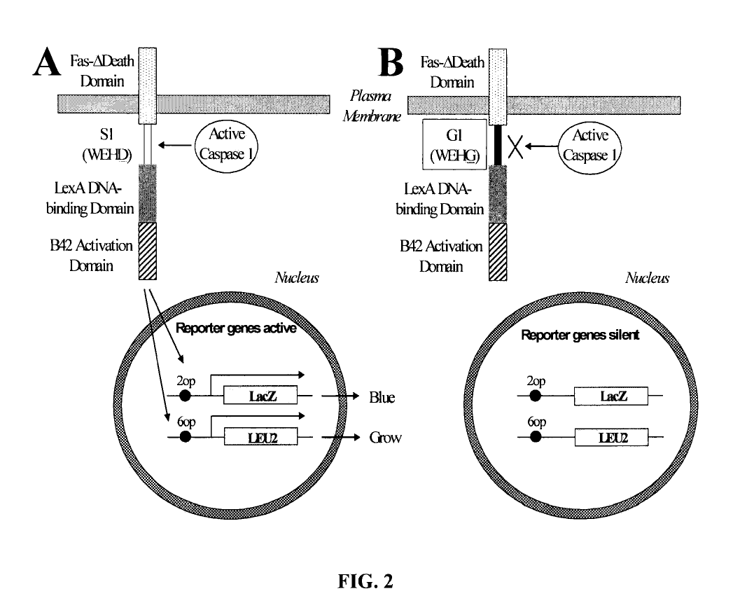

A reporter gene system for monitoring protease activity in living yeast was devised. For this purpose, a Type I transmembrane receptor (CD95; Fas) 4, most of its cytosolic domain was replaced by a chimeric transcription factor comprised of the DNA-binding domain of LexA and the transactivation domain of B42. Between Fas and the chimeric transcription factor (LexA-B42) was cloned various numbers of tetrapeptide sequences known to be recognized and cleaved by various members of the Caspase family. As a control, constructs were prepared in which the sessile aspartic acid within the tetrapeptide sequence was replaced with glycine (FIG. 2). These reporter constructs were expressed from plasmids in autotrophic yeast mutant strains EGY48 (MATα trp1, trp1, his3, ura3, LexA6op-LEU2) or EGY191 (MATα trp1, his3, ura3, LexA2op-LEU2), containing stably integrated LEU2 (allows grown on leucine-deficient media when the operator is activated by the transcriptional activator, LexA-B42) and lacZ (produces β-galactosidase) reporter genes under control of promoters containing 1, 2, 4, 6 or 8 copies of the LexA-binding operator (Estojak J, et al., Correlation of two-hybrid affinity data with in vitro measurements. Mol Cell Biol 1995; 15: 5820-9).

### A Genetic System for Monitoring Caspase 1 Activity in Yeast Cells.

The demonstration of a genetic system for monitoring Caspase-1 activity in yeast cells is shown in FIG. 2 consisting of 2A and 2B. (A) Engineered yeast were created that express a type-1 transmembrane protein (Fas-d-S1-TA) in which the Fas devoid of the death domain (Fas-d) is followed by a caspasel target site (S1-WEHD), and a transcriptional activator (TA-consisting of LexA DNA binding domain and B42 activation domain). LexA operators are located upstream of LacZ (2 operators) and LEU2 (6 operators) reporter genes, respectively. The cells expressing 6op-LEU2/2op-LacZ/TEF-Fas-d-S1-TA stimulate caspasel activity reporter, because expression of active caspase 1 (overexpressing a full-length caspase-1 construct) results in Fas-d-S1-TA cleavage at the caspase 1 target site (S1), releasing the transcription activator (TA), which enters the nucleus and activates LacZ and LEU2 reporter gene transcription. (B) A version of Fas-d-S1-TA in which the P1 aspartate is changed to glycine (Fas-d-G1-TA) cannot be cleaved by active caspasel. The cells expressing 6op-LEU2/2op-LacZ/TEF-Fas-d-G1-TA in which the glycine substitution is found serve as false-positive reporters for molecules that activate LacZ and LEU2 gene expressions independent of cleavage at the caspase 1 target site.

Caspases are initially produced as inactive zymogens, which typically undergo proteolytic processing to produce active enzymes composed in most cases of tetrameric assemblies with two large (˜20 kDa) and two small (˜10 kDa) subunits. To produce the active form of Caspases in yeast, each full-length Caspase construct was expressed (for Caspases-1, 2, 3, 4, 5, 6, 7, and 9) at high levels using plasmids with strong promoters, which results in ‘spontaneous” activation of these over-expressed proteases, generating the active, proteolytically processed forms of these proteases consisting large and small catalytic subunits. As for Caspases-8 and 10, however, a small amount of each full-length Caspase construct was expressed with a large amount of adaptor protein FADD to make the active forms, because large amounts of active Caspases-8 or -10 inhibited the yeast cell growth significantly. These active Caspases were then expressed in ΔLeu yeast with cleavable reporter constructs containing various tetrapeptide target sequences. Variables such as the strengths of the yeast promoters driving expression of the Fas-LexA-B42 cleavable fusion proteins to optimize signal:noise ratio were empirically adjusted, so that background (spontaneous) activation of the LEU2 and lacZ reporter genes was minimal (not shown), while titrating the number of LexA operators in the reporter genes to ensure a signal well above background, empirically determining that 2 LexA operators for lacZ and 6 LexA operators for LEU2 produced satisfactory results (not shown).

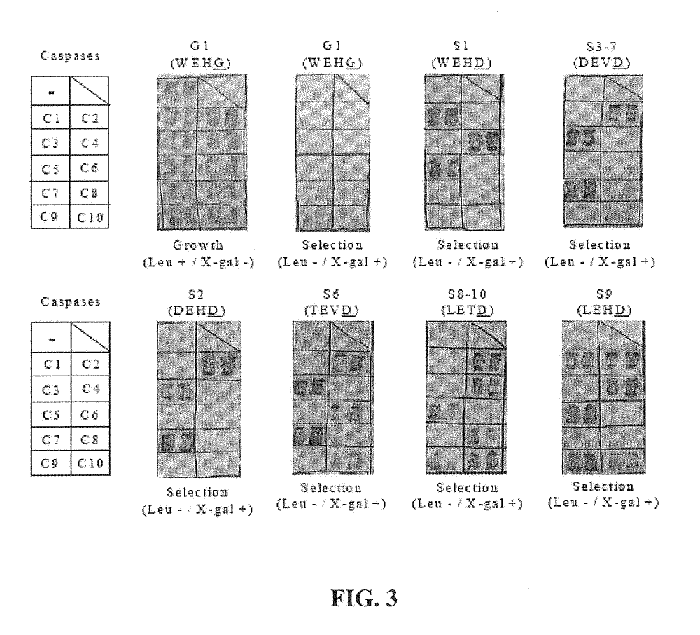

When plated on complete medium, all ΔLeu transformants grew, as expected (FIG. 3). However, a selective pattern of growth was observed for yeast transformants when they were plated on leucine-deficient medium to test activity of the LexAop-LEU2 reporter gene, along with X-gal substrate to colorimetrically detect the presence of β-galactosidase activity. In yeast containing the WEHG control sequence, which lacks the sessile aspartic acid required for Caspase cleavage, none of the yeast transformants grew or showed β-galactosidase-positivity, including Caspase-1 through Caspase-10. Yeast in which the Fas-LexA-B42 fusion protein contains the WEHD tetrapeptide sequence grew on leucine-deficient media and showed β-galactosidase-positivity when co-expressed with active Caspases-1, 4, and 5, but not Caspases-2, 3, 6, 7, 8, 9, or 10, perfectly matching predictions of prior studies in which the optimal tetrapeptide substrate sequences were determined for these human Caspases (Thornberry N A, et al. A combinatorial approach defines specificities of members of the caspase family and granzyme B. J Biol Chem 1997; 272: 17907-11).

Similarly, yeast containing the DEVD linker in the Fas-LexA-B42 fusion protein grew on leucine-deficient media and showed β-galactosidase-positivity when co-expressed with active Caspase-3 or Caspase-7, but not Caspases-1, 4, 5, 6, 8, 9, or 10, showing striking preferentiality among Caspases for cleavage of this reporter protein. Caspase-2 also cleaved the DEVD-containing reporter protein, which differs only slightly from the reported optimal substrate sequence of DEHD (Thornberry N A, et al. B. J Biol Chem 1997; 272: 17907-11). DEHD produced results very similar to DEVD, consistent with prior substrate specificity experiments suggesting that the P2 position where H was placed is the most flexible for Caspases-3 and -7 (Thornberry N A, et al. B. J Biol Chem 1997; 272: 17907-11). The other tetrapeptide sequences optimized for Caspase-6 (TEVD), Caspases-8/10 (LETD) and Caspase-9 (LEHD) resulted in less specific patterns of reporter protein activation, but nevertheless showed selectivity. For example, the “inflammatory” Caspases (Caspases-1, 4, and 5) did not activate the Caspase-6 substrate (TEVD). Also, the downstream effector Caspases (Caspases-3, 6, 7) did not activate the reporter proteins containing tetratpeptide sequences optimized for upstream initiator Caspases (LETD, Caspases-8/10; LEHD, Caspase-9).

### Substrate Specifities of the Caspases Expressed in Yeast.

As shown in FIG. 3 versions of Fas-d-S1-TA (Fas-d-G1-TA, Fas-d-S2-TA, Fas-d-S3-TA, Fas-d-S6-TA, Fas-d-S8-TA, and Fas-d-S9-TA) were created by substituting the caspasel cleavage site (S1-WEHD) with the pseudo-site (G1-WEHG), the caspase2 cleavage site (S2-DEHD), the caspase3 cleavage site (S3-DEVD), the caspase6 cleavage site (S6-TEVD), the caspase8 cleavage site (S8-LETD), and the caspase9 cleavage site (S9-LEHD), respectively, and were expressed with the LacZ and LEU2 reporter genes. The resultant yeast strains EGY48 expressing 6op-LEU2/2op-LacZ/TEF-Fas-d-G1-TA, 6op-LEU2/2op-LacZ/TEF-Fas-d-S1-TA, 6op-LEU2/2op-LacZ/TEF-Fas-d-S2-TA, 6op-LEU2/2op-LacZ/TEF-Fas-d-S3-TA, 6op-LEU2/2op-LacZ/TEF-Fas-d-S6-TA, 6op-LEU2/2op-LacZ/TEF-Fas-d-S8-TA, or 6op-LEU2/2op-LacZ/TEF-Fas-d-S9-TA, were transformed with caspase expression plasmids. Substrate specificities were determined for the ten caspases (caspases1-10, C1-C10). If cells express the active caspases with the suitable cleavage sites, the cells can grow in the selection medium (without leucine) and they hydrolyze X-gal to become blue. Expression of the Caspases (p424-ADH-Caspase1-FLAG, p424-ADH-HA-Caspase2, p424-ADH-Caspase3, p424-TEF-Caspase4, p424-ADH-Caspase5, p424-TEF-HA-Caspase6, p424-ADH-Caspase7, p424-CYC1-Caspase8-HA/TEF-HA-FADD, p424-ADH-HA-Caspase9-FLAG, and pCYC1-Caspase10-HA/TEF-HA-FADD) had no effects on the cell growth when plated on regulate (leucine-containing) medium.→

Taken together, these data demonstrate the performance of a one-component cleavable reporter gene system for measuring activity of the human Caspases in yeast. This one-component system was further used for screening human cDNA libraries expressed in yeast plasmids, where the cleavable Fas-LexA-B42 fusion protein was expressed without a Caspase, screening cDNA libraries for proteases that activate the reporter gene. Using the WEHD-containing reporter protein, eleven cDNAs that confirmed positive on repeated testing were obtained, including five that encoded Caspase-1 and six encoding Caspase-4. Using the DEVD-containing reporter, twelve clones that confirmed positive were obtained, including three that encoded Caspase-3 and nine encoding Caspase-7. These cDNA library screening results further validated the cleavable reporter system.

### Development of Two-component Systems Demonstrating Function of Caspase Activators in Yeast

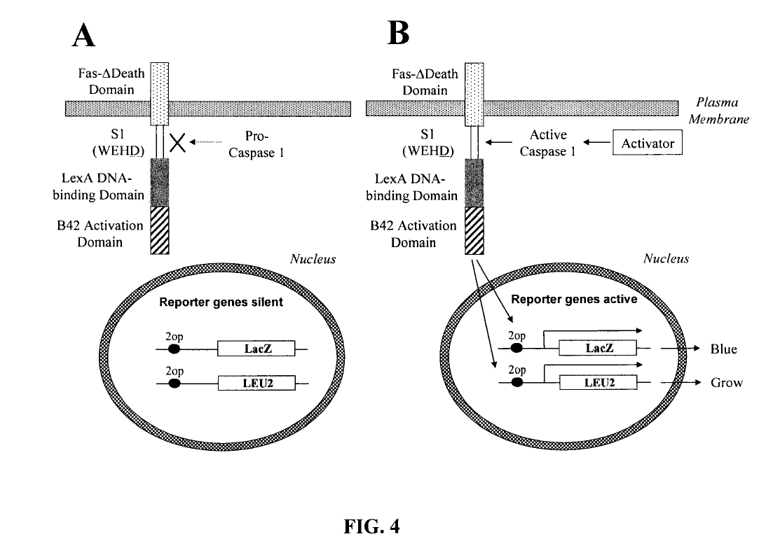

Two-component systems for assaying Caspase activity in yeast, in which the inactive proforms of the Caspases were co-expressed with various activator proteins were also created. FIG. 4 depicts the concept for Caspase-1, showing its preferred substrate WEHD.

### Schematic Representation of a Two-component System for Caspase1 Activators in Yeast Cells.

The schematic representation of a two-component system for caspase I activators in yeast cells is shown in FIG. 4 consisting of 4A and 4B (A) The zymogen (inactive) pro-caspase-1 protein is expressed in yeast, with the cleavable reporter containing the S1 site (WEHD), by regulating the expression level (p413-TEF-Fas-d-S1-TA/DTEF1-Caspase1-FLAG) in the yeast cell (EGY191 expressing 2op-LEU2/2op-LacZ). Under these conditions the reporter genes were silent. (B) Co-expression of ASC at high levels from p424-TEF-HA-Asc activated Caspase1, resulting in S1 site cleavage, and the transcription factor was released from the membrane to enter the nucleus and activate reporter genes.

Among the two-component systems interrogated were combinations of upstream initiator Caspases co-expressed with known activators, including: (1) pro-Caspase-1 plus ASC; (2) pro-Caspase-2 plus RAIDD; (3) pro-Caspase-9 plus a gain-of-function mutant of Apaf1; (4) pro-Caspase-8 plus FADD; and (5) pro-Caspase-10 plus FADD. In each of these cases, the upstream initiator Caspase, contains a N-terminal pro-domain (either CARD or DED) that binds a compatible CARD or DED in the activator protein. A two-component systems involving an upstream and downstream protease was also tested. For example, Caspase-9 was previously known as a direct upstream activator of downstream proteases, Caspases-3 and -7 (Slee E, et al. Ordering the cytochrome c-initiated caspase cascade: Hierarchic activation of caspases-2,-3,-6,-7,-8 and -10 in a caspase-9-dependent manner. J Cell Biol 1999; 144: 281-92). Active Caspase-9 was over-expressed in combination with the inactive proforms of Caspase-3 or -7, expressed at low levels. In each case, an appropriate cleavable reporter protein was co-expressed with the two-component systems, in yeast containing LEU2 and lacZ reporter genes. For all pair-wise combinations of pro-Caspase and upstream activator, the pro-Caspase was expressed in a relatively small amount using a low-copy plasmid containing CEN/ARS (1 to 2 copies per cell), and the upstream activator in a large amount using 2μ origin-containing plasmids (20 to 50 copies per cell) with relatively strong promoters, TEF, or ADH. Each pair of pro-Caspase and upstream activators were empirically adjusted by manipulating the number of LexA operators driving the LEU2 or lacZ reporter genes to achieve an acceptable signal:noise ratio. For example, for the combination of pro-Caspase-1 and ASC, it was determined that 2 LexA operators for LEU2 were superior to 4 or 6 operators (FIG. 4).

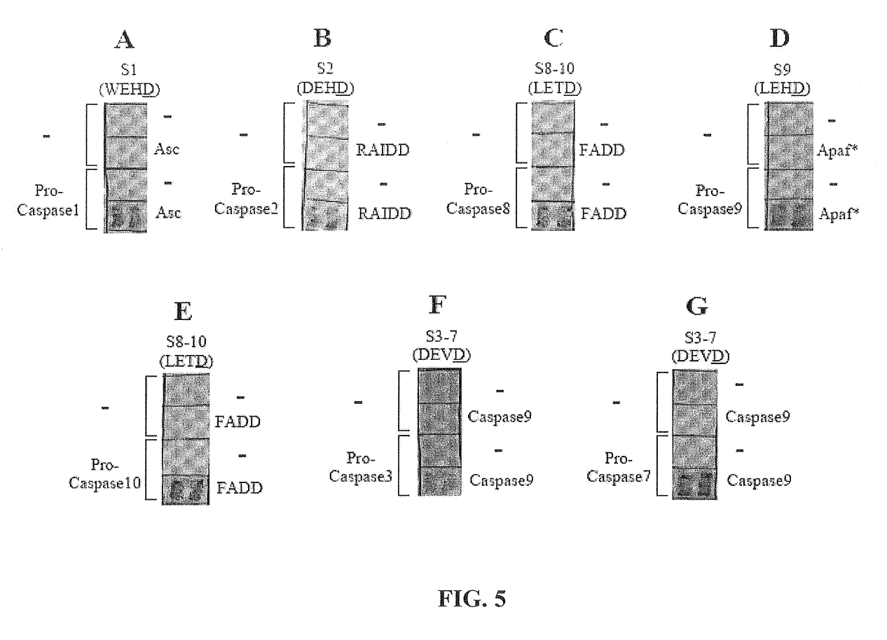

Successful activation of the cleavable reporter protein was achieved for each two-component system (FIG. 5). Expression of either the pro-Caspase alone or the upstream activator alone failed to result in activation of the LEU2 and lacZ reporter genes, as determined by ability to grow on leucine-deficient plates and β-galactosidase-positivity, confirming that both components are necessary for activating the reporter proteins. Moreover, pairing pro-Caspases with the wrong activators (e.g. Apaf1*+pro-Caspase-8; ASC+pro-Caspase-9) also failed to activate the reporters (not shown). Taken together, these data demonstrate the utility of yeast-based protease screening using two-component systems. Two-component systems for caspase activation in yeast.

As shown in FIG. 5 consisting of FIGS. 5A-5G yeast transformants were plated on leucine-deficient medium containing X-gal. For controls, the “empty” version of the plasmid was always introduced so that cells were subjected to identical selection conditions. (A) The recipient yeast strain EGY191 contained 2op-LEU2/2op-LacZ/TEF-Fas-d-S1-TA, with or without plasmids encoding pro-Caspase-1 (DTEF1-Caspase1-FLAG) or ASC (p424-TEF-HA-Asc), and cleaved the S1 site, because Asc itself did not cut the S1 site. (B) The recipient yeast strain EGY48 contained 6op-LEU2/2op-LacZ/DTEF2-Fas-d-S2-TA, with or without plasmids encoding pro-Caspase-2 (DGPD1-HA-Caspase2-FLAG) or RAIDD (p424-TEF-HA-RAIDD). (C) The recipient yeast strain EGY48 contained 6op-LEU2/2op-LacZ/GPD-Fas-d-S8-TA, with or without plasmid encoding pro-Caspase-8 (CYC1-Caspase8-HA) or FADD (p424-TEF-HA-FADD). (D) The recipient yeast EGY48 contained 6op-LEU2/2op-LacZ/TEF-Fas-d-S9-TA, with or without plasmids encoding pro-Caspase-9 (pTEF-HA-Caspase9) or an active mutant of Apaf-1 (p424-TEF-HA-Apaf*). (E) The recipient yeast strain EGY48 contained 6op-LEU2/2op-LacZ/GPD-Fas-d-S8-TA, with or without plasmids encoding pro-Caspase-10 (ADH-Caspase10-FLAG) or FADD (p424-TEF-HA-FADD). (F, G) The recipient yeast EGY191 contained 2op-LEU2/2op-LacZ/DTEF3-Fas-d-S3-7-TA, with or without plasmids encoding Caspase-9 (p424-ADH-HA-Caspase9-FLAG) and pro-Caspase-3 (CYC2-Caspase3) or pro-Caspase-7 (p424-ADH-Caspase7).

### Development of a Plural-component System—Mammalian Protease Activating Pathways Reconstituted in Yeast

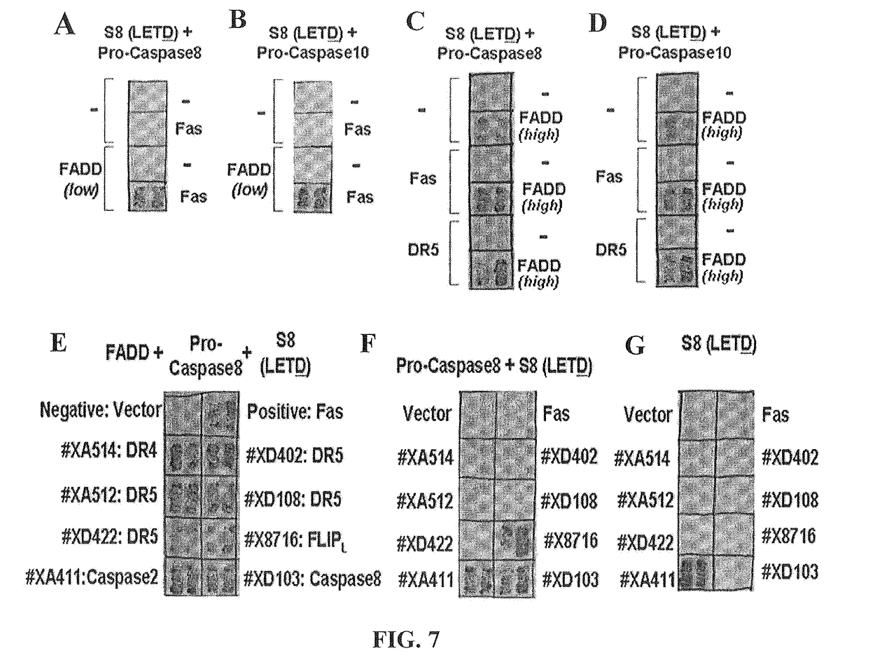

An increase in the complexity of reconstituted Caspase-activating systems in yeast, was created utilizing a plural component network comprising three or more components. Two different mammalian Caspase-activating networks were explored. In the first, the proximal portion of the extrinsic pathway was recapitulated, expressing (1) death domain (DD)-containing TNF-family death receptor, Fas [CD95]; (2) bipartite adapter protein FADD, which contains DD and DED; and (3) the proform of a DED-containing protease, either Caspase-8 or Caspase-10, along with (4) a cleavable reporter protein containing the Caspase-8/10 substrate tetrapeptide LETD. Two strategies for reconstituting the Fas/FADD/pro-Caspase-8 network in yeast were compared. First, we expressed the bridging adapter protein FADD at low levels using a constitutive but weak promoter such that the amount of FADD was inadequate to achieve pro-Caspase-8 activation in the absence of Fas. For that system, Fas was expressed at high levels so that it could oligomerize with itself without requiring Fas Ligand confirm (FIGS. 7A, and 7B). Second, low levels of pro-Caspase-8 and high levels of FADD (FIGS. 7C, and 7D) were expressed.

Additionally a 4-component system was reconstituted in yeast for γ-secretase activity, comprised of Presenlin-1, Nicastrin, APH-1, and PEN-2, using lacZ (β-galactosidase) reporter gene activation by a membrane-tethered cleavable transcription factor (Amyloid β-protein precursor [APP] fused to GAL1 transcription factor) as an endpoint for assessing activity of this transmembrane protease complex.

### Schematic Representation of Plural-component Systems for Caspase-8 Activation in Yeast.

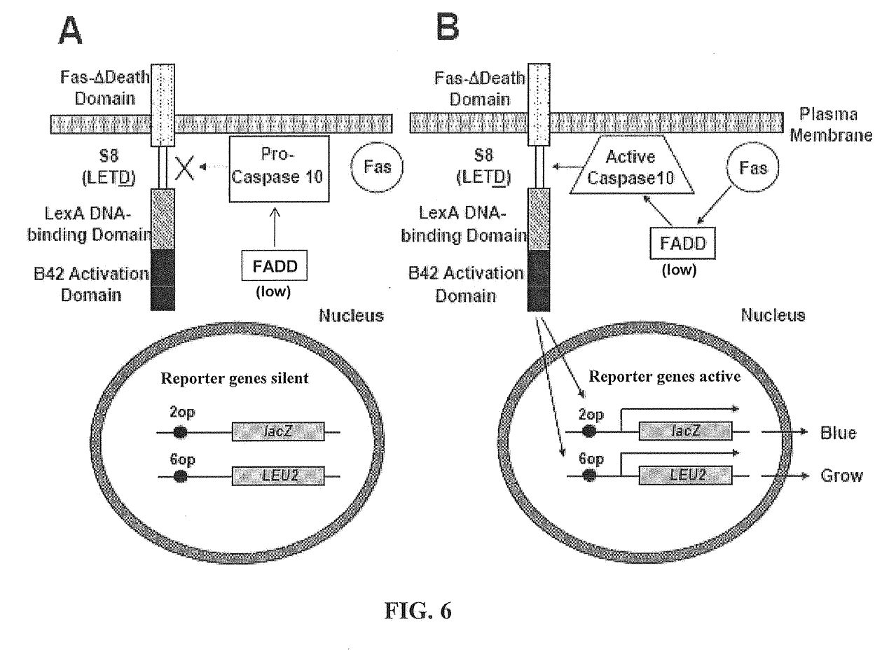

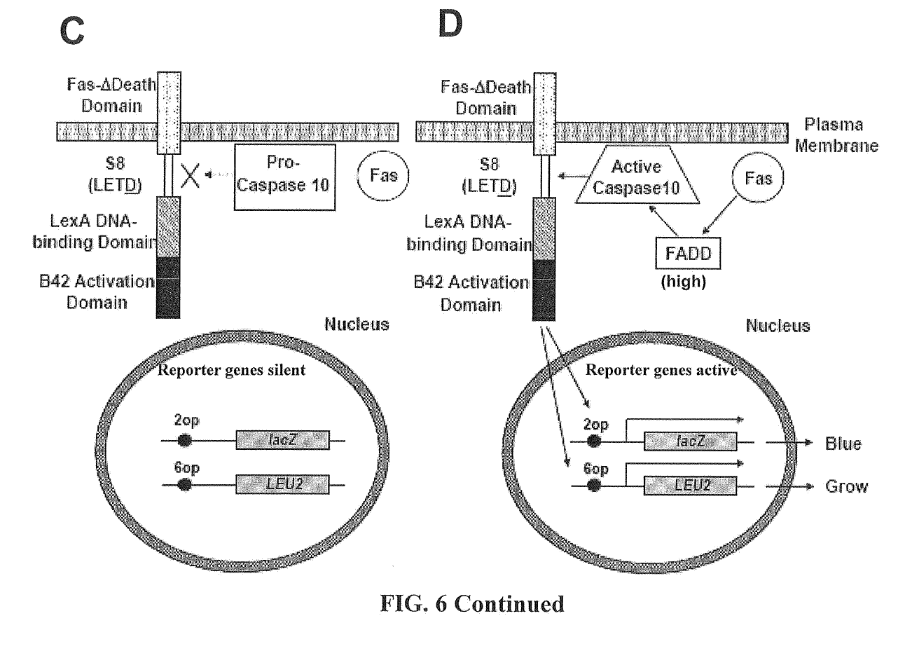

The schematic representation of a plural-component system for caspase-8 activators in yeast cells is shown in FIG. 6 consisting of FIGS. 6A-D (A) The zymogen pro-Caspase8 (from plasmid CYC1-Caspase8-HA) was expressed with substrate containing the S8-10 cleavage element (from plasmid p413-TEF-Fas-d-S8-TA) in the yeast EGY48 expressing 6op-LEU2. A small amount of FADD was also expressed without activating the Caspase-8 from plasmid p426-2op-LacZ/DADH1-FADD. (B) Addition of FADD-binding protein Fas, expressed at high levels from plasmid p424-Fas) activated Caspase-8, releasing the transcription factor. (C, D) In an alternative approach, FADD expressed under control of an inducible plasmid, using p424-ADH-HA-FADD, thus completing the connection between constitutively expressed pro-Caspase-8 and Fas.

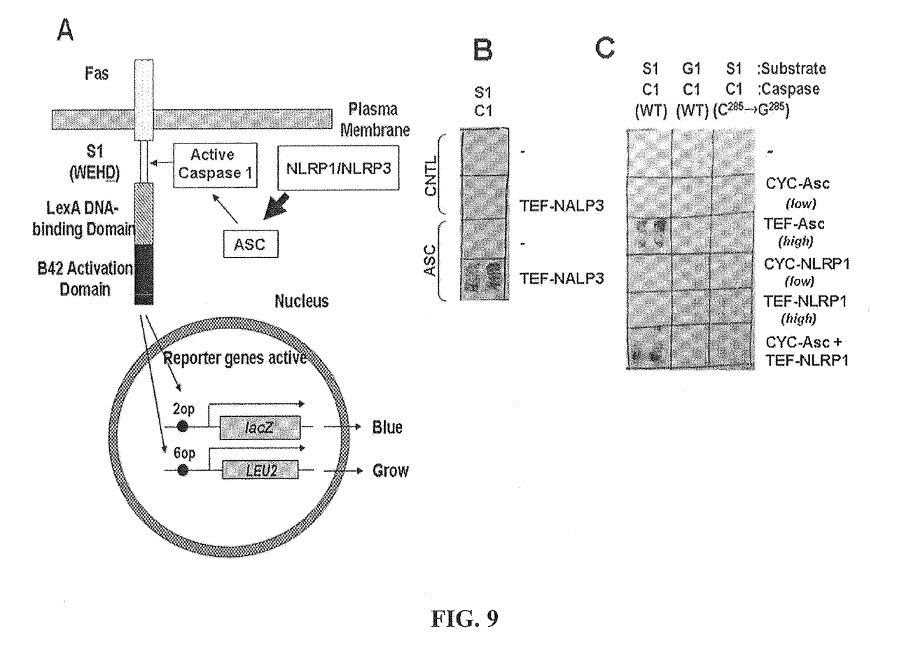

By empirically comparing different strength promoters for driving expression of the three components and adjusting the number of LexA operators in the promoters of the LEU2 and lacZ reporter genes, the determination of conditions that produced the desired result for the approach was reached whereby low levels of constitutive FADD were complemented by expression of Fas confirm(FIGS. 9A and 9B). The alternative approach resulted in Fas-independent activation of Caspase-8 and -10, though the reactions were enhanced by Fas or other death receptors (e.g. DR5).

### Testing of Yeast-based, Plural-component Systems for Activating Caspases-8 or -10.

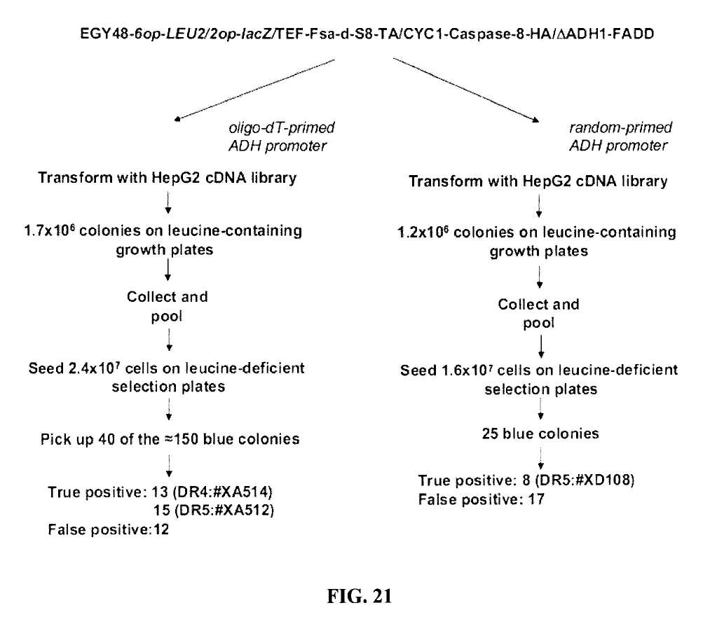

The results of testing yeast-based, plural-component systems for activating caspases-8 or -10 are shown in FIG. 7 consisting of FIGS. 7A-G. (A, B) Yeast EGY48 cells expressing Caspase-8/-10-cleavable substrate from 6op-LEU2/2op-LacZ/TEF-Fas-d-S8-TA, a n d either pro-Caspase-8 or pro-Caspase-10 (from CYC1-Caspase8-HA or CYC1-Caspase10- FLAG), and FADD from a plasmid with a weak promoter were transformed with an empty vector (p424-ADH) or a plasmid expressing Fas (p424-ADH-Fas). The small amount of pro-Caspase-8 was not activated by the small amount of FADD, unless Fas was present, resulting in cell growth with blue color when transformed cells were grown on medium lacking leucine and containing X-gal substrate. (C, D) Yeast EGY48 cells expressing Caspase-8/-10-cleavable substrate, low levels of either pro-Caspase-8 or -10, and either Fas or DR5 were transformed with an empty vector p424-ADH or the same plasmid expressing FADD (p424-ADH-HA-FADD) from a strong promoter. In the absence of FADD, the small amount of pro-Caspase-8 was not activated and did not cut the S8 site of the Fas-d-S8-TA substrate, but when FADD was supplied, then substrate cleavage was achieved. However, Caspase-8 and -10 activation were Fas/DR5-independent (constituting a two-component but not plural or three-component system) but the reactions were enhanced significantly by expressing Fas or DR5. Examples of cDNA cloning results. Yeast strain EGY48 containing 6op-LEU2 and 2op-lacZ reporter genes and expressing the LETD-containing substrate (expressed from pTEF-Fas-d-S8-TA) with or without pro-Caspase-8 (CYC1-Caspase8-HA), and/or FADD (ΔADH1-FADD). Cells were subsequently transformed with various cDNA libraries (See FIGS. 21-23) and clones that activated the reporter genes were characterized by recovery of cDNA library plasmids and retransformation into yeast expressing FADD and pro-Caspase-8 (E), pro-Caspase-8 without FADD (F), or neither (G). Among the positive clones were #XA514 (n=13) encoding a fragment of DR4 (G210-E468), #XA512 (n=15) encoding a fragment of DR5 (A43-S411), #XD108 (n=8) encoding a fragment of DR5 (Y99-S411), and #XD422 (n=3) encoding a fragment of DR5 (A192-S411), and #XD402 (n=7) encoding a fragment of DR5 (V124-S440), all of which activated the lacZ reporter gene in cells containing FADD and Caspase-8 (E) but not in cells lacking FADD (F) or both (G). Also obtained were cDNAs #XD103 encoding a fragment of Caspase-8 (M1-R233), #X8716 encoding a full-length FLIPL, and #XA411 encoding a full-length Caspase-2. Assays were performed in duplicate, growing cells for 4 days on plates.

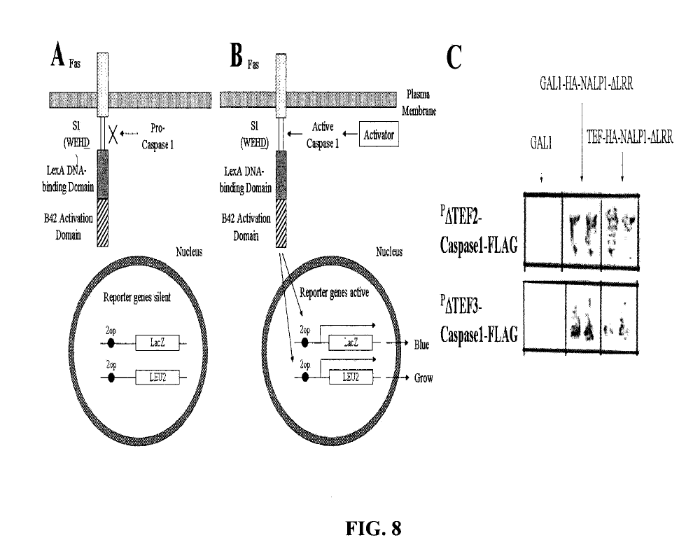

Another plural-component Caspase-activating network was similarly created, using variations on the same approach. Specifically, the pro-inflammatory plural-component system was reconstituted in yeast and comprised: (1) the CARD-containing protease, pro-Caspase-1; (2) the bipartite adapter ASC, which contains CARD and PYD domains; and (3) the NLR-family protein NALP1, using a gain-of-function mutant of NALP1 that is constitutively active without requiring a bacterial ligand to induce its oligomerization (Faustin B, et al. Reconstituted NALP1 inflammasome reveals two-step mechanism of Caspase-1 activation. Molecular Cell 2007; 25(5): 713-24). For this system, the reporter protein contained the Caspase-1 cleavage sequence, WEHD (FIG. 8). Pro-Caspase-1 and adapter protein ASC was expressed in yeast under constitutive promoters, using a weak promoter for ASC that failed to activate pro-Caspase-1 in the absence of other cofactors. The gain of function mutant NALP1(ΔLRR) was expressed under control of an inducible promoter. Upon induction of NALP1(ΔLRR) expression, the reporter genes became activated. Exclusion of any of the three components of this system (NALP1, ASC, or pro-caspase-1) prevented reporter gene activation (not shown). It was determined that NALP1ΔLRR can activate pro-Caspase-1 in yeast in an ASC-dependent manner, thus demonstrating reconstitution in yeast of another plural-component system for Caspase activation.

### Yeast Assay for NALP1.

The results of a yeast assay for NALP1 are shown in FIG. 8 consisting of FIGS. 8A-C. (A, B) The basis for the cleavable reporter assay is depicted. The membrane tethered transcription factor consists of the DNA-binding domain of the LexA protein and the transactivation domain of the B42 protein, fused to a peptide linker with caspase-1 cleavage sequence (WEHD) and the extracellular and transmembrane domains of Fas (CD95). In the absence of caspase-1 activators, the reporter remained uncleaved and membrane anchored (A). When caspase-1 was activated, then the reporter was cleaved, releasing the chimeric LexA/B42 transcription factor to leave the membrane and enter the nucleus, where it induced expression of LEU2 and LacZ genes (B). (C) An example of the assay results was shown. Yeast were plated on selective media and β-galactosidase activity was ascertained by a calorimetric assay. Plasmids employed were empirically optimized. The example shown employed EGY48 strain yeast, which contain a LacZ reporter gene containing two copies of the LexA operator and in which ASC expression was driven from a plasmid containing a constitutive promoter with weak activity (CYC1). Two different strength promoters were tried for expressing pro-caspase-1: PΔTEF2 (top) and PΔTEF3 (bottom), both of which worked well for this assay. The yeast cells were transformed with a GAL1 promoter-driven plasmid containing no cDNA insert (left) or a cDNA encoding NALP1ΔLRR (middle). Alternatively, a plasmid with TEF promoter driving expression of NALP1ΔLRR was employed (right).

### Yeast Assay for NLRs.

The results of a yeast assay for NLRs are shown in FIG. 9 consisting of FIGS. 9A-C. (A) Yeast strain EGY48 containing 6op-LEU2/2oplacZ expressing pro-Caspase-1 (driven by ΔTEF3 promoter, which expresses at low levels) with membrane tethered transcription factor substrate containing WEHD peptide linker (S1 substrate) were transformed with plasmids encoding adapter protein Asc (expressed at low levels from a CYC promoter) and upstream activators either NLRP1ΔLRR or NLRP3ΔLRR (expressed at high levels from TEF or GAL1 promoters). When Caspase-1 is activated via the combination of Asc and NLPR1 or NLRP3, the reporter is cleaved, releasing the chimeric LexA/B42 transcription factor to leave the membrane and enter the nucleus, where it induces expression of LEU2 and lacZ genes. (B) EGY48 cells (6op-LEU2, 2op lacZ) were transformed with plasmids encoding the S1 substrate, pro-Caspase-1 (C1) expressed from ΔTEF promoter, Asc (low) from CYC promoter, and NLRP3ΔLRR from TEF promoter. Control transformants received the corresponding empty plasmids (−). Cells were grown for 2 days on leucine deficient plates containing X-gal and galactose (to induce NLRP3ΔLLR expression). (C) The EGY48 recipient (6op-LEU2, 2op-lacZ) yeast cell strain contained plasmids encoding substrates with either the WEHD tetrapeptide cleavable linker (S1 substrate expressed from TEF-Fas-d-S1 (WEHD)-TA transcriptional unit) or WEHG non-cleavable substrate (G1 substrate expressed from TEF-Fas-d-G1 (WEHG)-TA) in plasmids encoding either wild-type (“C1/WT”) or Cys285 mutated Caspase-1 (“C285→G285”) (expressed from pΔTEF1-Caspase-1-FLAG or ΔTEF1-Caspase-1 (C285→G285)-FLAG transcriptional units). Cells were transformed with the plasmids expressing a small amount of Asc (CYC-Asc), a large amount of Asc (TEF-Asc), a small amount of NLRP1ΔLRR (CYCNLRP1), a large amount of NLRP1ΔLRR (TEF-NLRP1), various combinations, or empty vectors (−). Yeast transformants were plated on leucine-deficient medium containing Xgal. The large amount of Asc (expressed from TEF promoter) activated Caspase-1 by itself. NLRP1ΔLRR was unable to activate Caspase-1 by itself, albeit expressed at high levels (confirmed by immunoblotting [not shown]). NLRP1ΔLRR activated pro-Caspase-1 in the presence of a small amount of Asc, under conditions where the amount of Asc expressed (from CYC promoter) was insufficient to independently activate Caspase-1.

### HTS Implementation of Yeast-based Protease Network Reconstitution Assays.

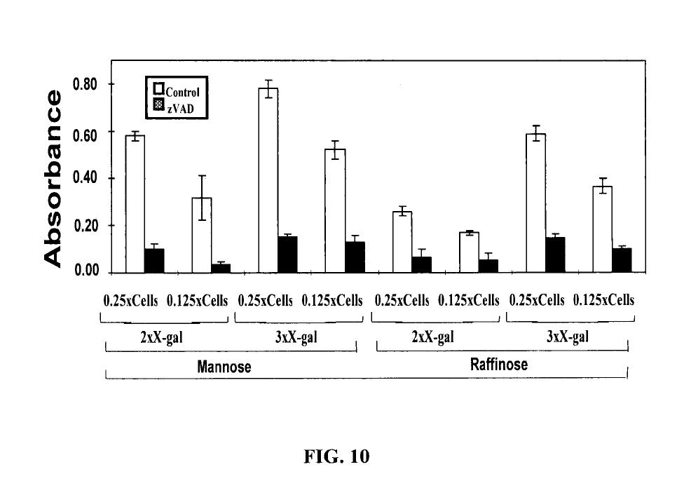

When converting the yeast-based protease reporter system to HTS assay, conditions from agar plates to liquid medium in microtiter wells must be revised. This revision comprises, utilizing the measured β-galactosidase produced by yeast carrying the Caspase-cleavable reporter proteins, and assaying the colorimetric product (OD620ₙₘ) derived from X-gal substrate in 384 well plates, as a measure of the lacZ reporter gene activity. We compared β-galactosidase activity produced in the presence or absence of the broad-spectrum caspase inhibitory compound, zVAD-fmk (benzoyl-Valinyl-Alaninyl-Aspartyl-fluoromethylketone). Among the variables that were initially interrogated were initial seeding cell density, time of culture, concentration of X-gal substrate, and supporting carbohydrate (raffinose vs mannose). Note that it was determined that it is not advisible to employ glucose in yeast media because it represses the GAL1 promoter used in some plasmids. An example of data obtained using the plural-component system of Fas/FADD/Caspase-8 is presented. All variables tested altered the signal:noise ratio of the assay. For the Fas/FADD/Caspase-8 plural-component assay, the best results were achieved with ˜2×10⁵ cells per well starting density cultured for ˜3 days in raffinose-containing media with 4× concentration of X-gal substrate (FIG. 10). For the two-component system of ASC/Caspase-1 and for the plural-component system of NALP1ΔLRR/ASC/Caspase-1, the same conditions were optimal, among those tested (not shown). For the Fas/Fadd/Caspase 8, the caspase and adaptor protein (Fadd) were expressed at relatively small amounts (a weak promoter and low copy number plasmid are used) and the upstream activator (Fas) was expressed in a large amount.

Optimization of Signal:noise Ratio in Microtiter Plates.

As shown in FIG. 10 EGY48 cells containing the plural-component Fas/FADD/Caspase-8 system and Caspase-8-cleavable reporter were grown in 384 well plates, comparing cell densities, X-gal concentrations, and sugar additives (mannose vs raffinose). The activity of β-galactosidase was measured after 72 hrs culture (mean+std dev; n=3), for cells grown in the absence (white bars) or presence (black bars) of 100 μM zVAD-fmk Caspase inhibitor.

The 384 well assay format was further validated, using zVAD-fmk, a peptidyl inhibitor with broad-spectrum activity against animal Caspases. For some experiments, a Calpain inhibitor was employed in side-by-side experiments as a control. The broad-spectrum Caspase inhibitor zVAD-fmk inhibited β-galactosidase activity in a dose-dependent fashion, with the concentration required for achieving 50% inhibition ranging from ˜1-10 μM in these assays (FIG. 10). In contrast, the Calpain inhibitor did not suppress activation of the lacZ reporter gene. These experiments verified that the 384 well version of the yeast-based Caspase-dependent reporter gene assay faithfully indicates the activity of an inhibitory compound, thus fulfilling one of the prerequisites for developing HTS assays.

### Validation of Yeast-based Caspase Assay Using Pharmacological Inhibitor of Caspases.

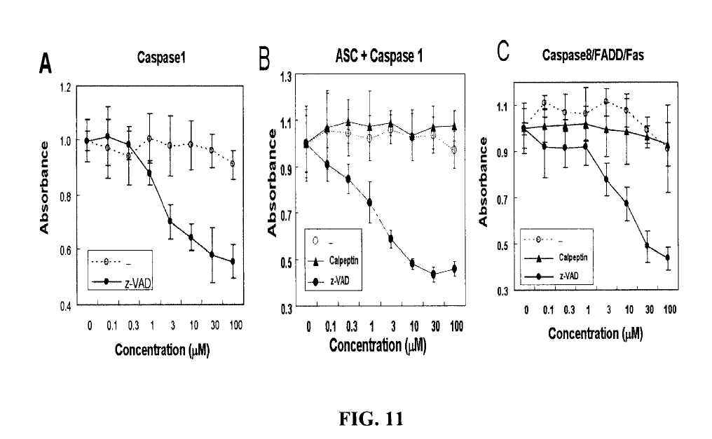

The validation results of a yeast-based Caspase assay using a pharmacological inhibitor of Caspases are shown in FIG. 11 consisting of FIGS. 11A-C (A) Caspase-1, single-component assay. Yeast cells expressing TEF-Fas-d-S1-TA/GPD-HA-Caspase1-FLAG were incubated for 3 days in selection media (SD/X-gal) with the indicated concentrations of z-VAD-fmk () or DMSO only (∘), using 384-well plates. (B) ASC/Caspase-1 two-component system. Yeast cells expressing TEF-Fas-d-S1-TA/DTEF1-Caspase1-FLAG/TEF-HA-Asc were incubated for 3 days in the selection media (SD/X-gal) in 384 well plates with the indicated concentrations of calpeptin (▴) or z-VAD-fmk () or an equivalent volume of DMSO was added (∘). (C) Fas/FADD/Caspase8 plural-component system. Yeast cells expressing TEF-S8-TA/CYC1-caspase8-HA with a small amount of FADD and a large amount of Fas, were incubated in selection media (SD/X-gal) with the indicated concentrations of calpeptin (▴) or z-VAD-fmk (), or equivalent volume of DMSO (∘), in 384-well plates for 3-4 days. For (A-C), relative absorbance at 620 nm was measured (mean ±std dev; n=3-5 independent experiments). (* indicates p<0.001).

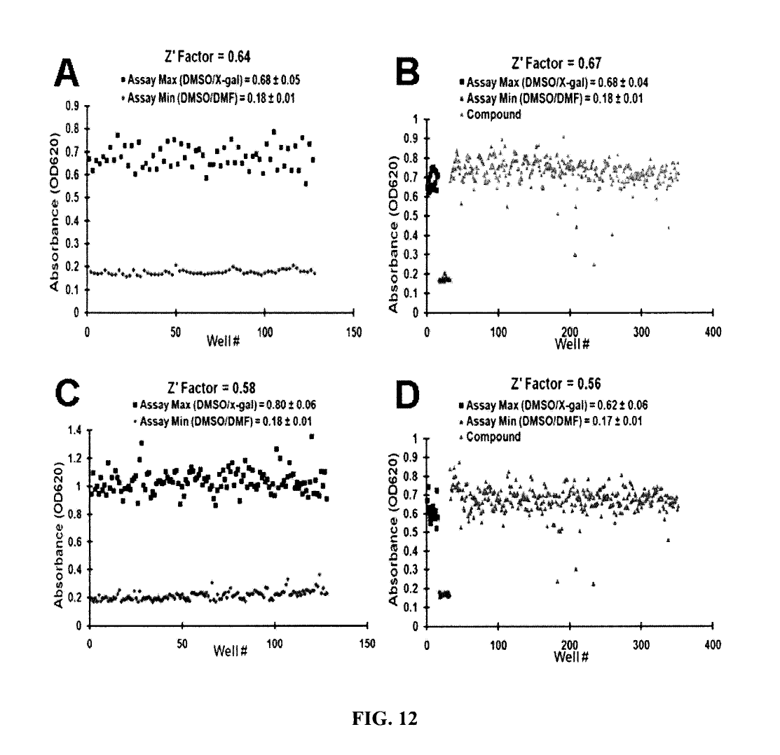

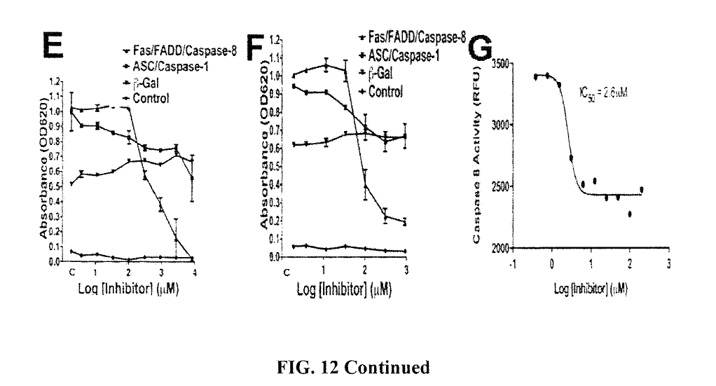

To assess the reproducibility of the 384 well microplate assay, multiple replicate wells in 384 well plates were prepared representing the negative (assay max) and positive (assay min) controls for the assays. For the plural-component system of NALP1ΔLRR/ASC/Caspase-1, the maximum signal was produced by cells grown with X-gal substrate, while the minimum was set by cells grown without X-gal substrate. As described above, for this assay, NALP1ΔLRR-mediated activation of Caspase-1 resulted in activation of the cleavable transcription factor, producing β-galactosidase, which was measured using a colorimetric substrate. The magnitude of the readout of the Z′ factor was optimized by empirically adjusting yeast cell density, incubation time, and other variables in ordere to employ a 384 well assay format. The Z′ factor for the assay was determined by reading multiple replicates of the assay max and min, and determined to be >0.6, and thus suitable for HTS (FIG. 12). A screen of 1280 compounds was performed (LOPAC library), achieving an acceptable average hit rate of ˜0.5 per 384 well plate. An example of primary screening results is shown for a 384 well plate in which 3 inhibitory compounds were identified (FIG. 12B).

### HTS Implementation of Yeast-based NALP1 Inflammasome Assay.

The results of a HTS of a yeast-based NALP1 inflammasome assay are shown in FIG. 12 consisting of FIGS. 12A-G (A) NALP1 yeast screen controls. Yeast cells containing ASC/Nalp1ΔLRR/Caspase1 were plated at 1.88×10⁵ cells/well in a 384 well plate in media containing 1% galactose/2% raffinose, with (squares) or without (diamonds) 400 μg/ml X-gal substrate. The plates were incubated for 72 hrs at 30° C. and the plates were read at OD620. (B) Yeast Screen of LOPAC compound library plate 4 for NALP1. Yeast cells containing ASC/NALP1/Caspase1 were plated at 1.88×105 cells/well in a 384 well plate in media containing 1% galactose, 2% raffinose, with or without 400 μg/ml X-gal. The plates were incubated for 72 hrs at 30° C. and the plates were read at OD620. The compounds were screened at 100 μM and “hits” were defined by >50% reduction (as shown). (C) Validation of HTS assay for Fas-FADD-Caspase-8 system. Yeast cells containing Fas/FADD/Caspase-8 were cultured in 384 well plates as above with (blue squares) or without (purple diamonds) Xgal substrate, the plates were incubated for 72 hrs at 30° C. and analyzed as above. (D) Example of library screening plate for Fas-FADD-Caspase-8. Yeast cells containing Fas/FADD/Caspase-8 were plated at ≈2×105 cells/wells in 384 well plates in Selection Media, without (purple triangles) or with (all others) 400 μg/ml X-gal (final concentration), and without (triangles, squares) or with (diamonds) compounds in 10% DMSO (˜10 μM final). Plates were incubated for 48 hrs at 30° C. and OD620 values recorded. “Hits” were defined by >50% reduction (shown as green diamonds). (E, F) Examples of compounds showing evidence of selective inhibition of Fas-FADD40 Caspase-8. EGY48 yeast cell lines employed for deconvoluting hits included yeast expressing lacZ gene (□) expresses LexA/B42 transcription factor from GAL1 promoter; cells grown in galactose media to induce promoter) to eliminate false-positives due to β-galactosidase inhibition and Asc/Caspase-1-expressing yeast (□) to eliminate hits that interfere with a different Caspase. Yeast containing empty vector were also included (□). Yeast cells (at 2×105 cells/well) containing Fas/FADD/Caspase-8 (▴) or other strains were cultured in 384 well plates in a total volume of 40 μL Selection Media, with 400 μg/ml X-gal final concentration, without (“C”) or with various concentrations of compound CID-5154 or CID-3101. The plates were incubated for 2-4 days at 30° C. and OD620 values recorded, (G) Example of compound showing evidence of inhibition of Caspase-8. An in vitro biochemical assay was used containing purified recombinant Caspase-8 and fluorigenic substrate Ac-IETD-AFC. Data represent relative fluorescence units (RFU) assessed in the absence or presence of various concentrations of compound (CID-2531).

Determination of IC50 Values for zVAD-fmk Inhibition of Caspases in Yeast.

Yeast expressing various Caspases alone (at high levels) or in combination with upstream activators (at low levels) and cleavable substrates containing appropriate tetrapeptides reorganized by these proteases were used in 384 well β-galactosidase activity assays to assess inhibition by zVAD-fmk. The compound was titrated into assays at various concentrations and percentage inhibition was determined. IC50 values were determined using PRIZM software for analysis with the results displayed in Table 2 below.

TABLE 2
IC₅₀ values for z-VAD inhibition of Caspases

| Enzyme | IC₅₀ (uM) |
|---|---|
| Caspase1 | 3.07 |
| Caspase1 activated with Asc | 1.00 |
| Caspase6 | 6.78 |
| Caspase7 | 4.11 |
| Caspase8 activated with FADD + Fas | 5.42 |
| Caspase9 activated with Apaf* | 10.66 |
| Caspase10 activated with FADD | 9.52 |

### Substrate Sequence- and Caspase Activity-dependent Cleavage of the S1, S2, and S9.

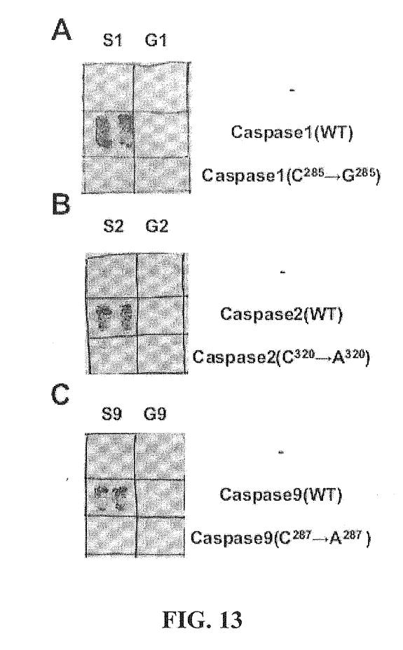

As shown in FIG. 13 consisting of 13A, 13B and 13C yeast transformants were plated on leucine-deficient medium containing X-gal. (A) The recipient yeast cell strains, EGY48-6op-LEU2/2op-lacZ/TEFFas-d-S1 (WEHD)-TA (S1), or EGY48-6op-LEU2/2op-lacZ/TEF-Fas-d-G1(WEHG)-TA (G1) were transformed with the plasmids encoding the active wild type Caspase1 (WT), the catalytically-defective Caspase1 (C285→G285), and the empty vector (−). (B) The recipient yeast cell strains, EGY48-6op-LEU2/2op-lacZ/TEF-Fas-d-S2(DEHD)-TA (S2), or EGY48-6op-LEU2/2op-lacZ/TEF-Fas-d-G2(DEHG)-TA (G2) were transformed with the plasmids encoding the active wild type Caspase2 (WT), the catalytically-defective Caspase2 (C320→A320), and the empty vector (−). (C) The recipient yeast cell strain, EGY48-6op-LEU2/2op-lacZ/TEF-Fas-d-S9(LEHD)-TA (S9), or EGY48-6op-LEU2/2oplacZ1 TEF-Fas-d-G9(LEHG)-TA (G9), were transformed with the plasmids encoding the active wild type Caspase9 (WT), the catalytically-defective Caspase9 (C287→A287), and the empty vector (−).

Flow Chart for cDNA Library Screening Using a Reporter Gene Strategy Based on Cleavable Transcription Factor.

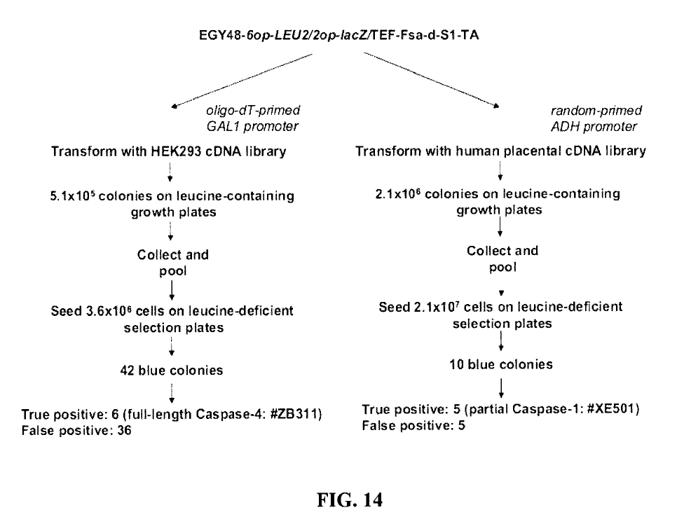

The example shown in FIG. 14 is S1 substrate. The yeast strain EGY48 containing 6op-LEU2 and 2op-lacZ reporter genes and Caspasecleavable substrate (expressed from TEF-Fas-d-S1-TA) was transformed (left) with a HEK293 cell cDNA library (oligo-dT-primed, PGAL1 promoter). Independent colonies of 5.1×105 clones appeared on growth plates in 48 hours. Cells were collected, pooled, and a portion (3.6×106) was seeded on leucine-deficient selection plates containing Xgal. Blue-colored colonies (n=42) appeared within a week, of which 6 corresponded to clone #ZB311 encoding full-length Caspase-4 (which is known to cleave WEHD tetrapeptide [Thornberry, N. A, et. al., J. Biol. Chem. 272, 17907-17911 (1997)]), while the rest were false positives. (Right) Another cDNA library screen (HEK293 random primed, ADH promoter used to drive expression) resulted in 2×106 clones, which were pooled and 2.1×107 cells were screened on leucine-deficient, X-gal-containing plates, resulting in 10 blue colonies. Five clones (including #XE501) encoding a fragment of Caspase-1 (L89-G403) were isolated. The remaining five clones were apparent false positives.

Flow Chart for cDNA Library Screening Using Reporter Gene Strategy Based on Cleavable Transcription Factor.

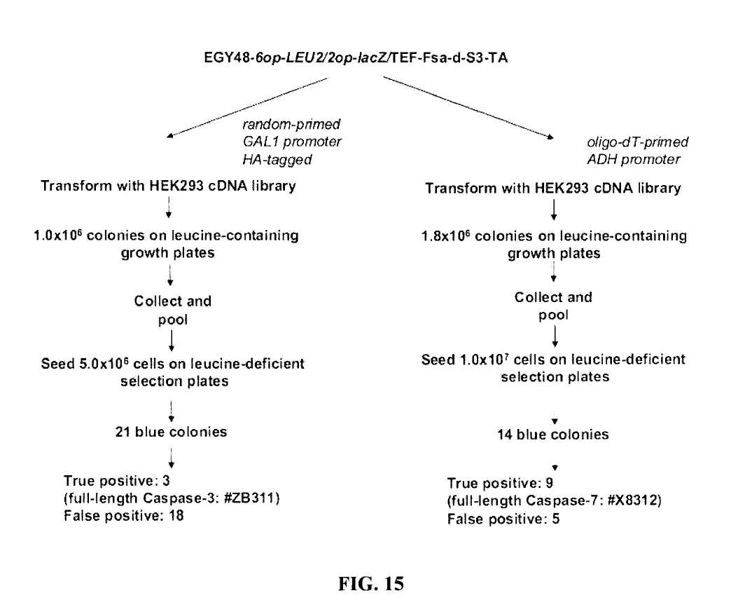

The example shown in FIG. 15 is S3/S7 substrate. Yeast strain EGY48 containing 6op-LEU2 and 2op-lacZ reporter genes and the DEVD containing cleavable transcription factor (expressed from TEF-Fas-d-S3-TA) was transformed with a HEK293 cell cDNA library (random-primed, PGAL1 promoter, HAtagged) (left). Independent colonies (˜1.0×106) appeared on growth plates within 48 hours. Cells were collected, pooled, and a portion (5.0×106) was seeded on the leucinedeficient selection plates containing X-gal. Blue-colored colonies (n=21) appeared within a week, of which 3 clones (including #ZB331) encoded full-length Caspase-3, while the rest were false positives. In a similar experiment (right), another HEK293 cDNA library (oligo-dT-primed, PADH promoter) (1.8×106 initial colonies) was screened, resulting in 9 positive clones (including #X8312) encoding full-length Caspase-7. The remaining five clones were false positives. Both cloned Caspases are known to cleave DEVD tetrapeptide [Thornberry, N. A, et. al., J. Biol. Chem. 272, 17907-17911 (1997)].

Use of One-component Yeast-based Caspase Activity Assay for cDNA Library Screening.

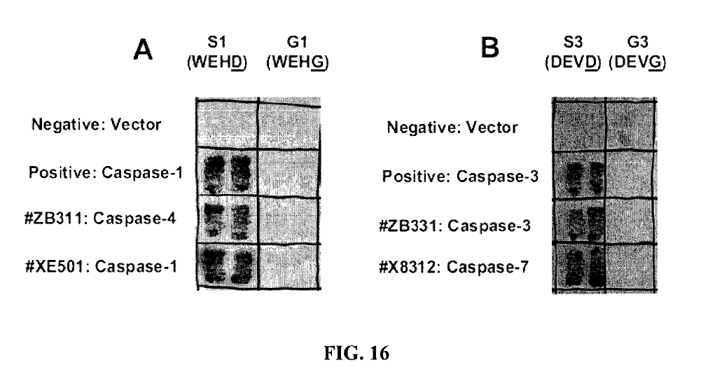

FIG. 16 consisting of 16A and 16B demonstrates the results of a one-componsnt yeast-based Caspase activity assay for cDNA library screening. (A) The plasmids containing library cDNAs from FIG. 14 were recovered and re-transformed into yeast cells containing 6op-LEU2 and 2op-lacZ reporter genes with either cleavable (WEHD-containing) (expressed from TEF-Fas-d-S1-TA) or non-cleavable (WEHG-containing) transcription factor substrate (expressed from TEF-Fas-d-G1-TA) to confirm whether they cleave S1 specifically. As controls, yeast were transformed with a plasmid encoding Caspase-1 (“Positive” control) or the empty vector (“Negative” control). Assays were performed in duplicate, with cells grown on selection plates for 4 days. Note that the cDNA library clones supported lacZ reporter gene activation only when co-expressed with the S1 cleavable substrate. (B) The cDNA library plasmids from FIG. S3 were recovered and used to re-transform the yeast strains containing either the same cleavable DEVD-containing or DEVG noncleavable transcription factor, expressed from TEF-Fas-d-S3-TA (“S3” substrate) and TEF-Fas-d-G3-TA (“G3” substrate), respectively. Assays were performed in duplicate and cells grown on selection plates for 4 days. Note that the two cDNA library clones activated the lacZ reporter gene only when co-expressed with cleavable S3 (DEVDcontaining) substrate. As controls, yeast cells were transformed with plasmids encoding Caspase-3 (“Positive” control) or the empty vector (“Negative” control).

### Validation of Yeast-based Assays for Effector Caspase Activators—Two and Three-component Systems.

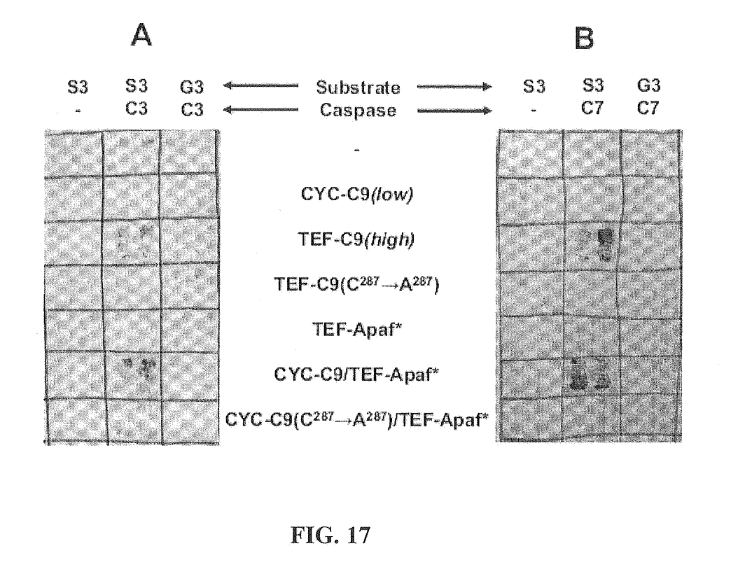

As shown in FIG. 17 consisting of 17A, and 17B yeast EGY191 strain containing 2op-LEU2/2op-lacZ or EGY48 strain containing 6op-LEU212op-lacZ were employed for developing assays for activators of downstream effector Caspases (e.g. Caspases-3 and -7). Yeast transformants were plated on leucine-deficient medium containing X-gal. Yeast were transformed with plasmids encoding membrane tethered transcription factor substrate with either DEVD-containing cleavable (S3) or DEVG-containing non-cleavable (G3) linkers, and with plasmids encoding pro-Caspase-3 (C3) or pro-Caspase-7 (C7) or the corresponding empty vector (−). (Note that the optimal tetrapeptide sequence for both Caspase-3 and Caspase-7 has previously been reported to be DEVD). Yeast transformants were as follows: (A) S3 Substrate/No Caspase: EGY191-2op-LEU2/2oplacZ/DΔTEF3-Fas-d-S3 (DEVD)-TA; S3 Substrate/Caspase-3: EGY191-2op-LEU2/2oplacZ/DΔTEF3-Fas-d-S3/7(DEVD)-TA/DΔCYC2-HA-Caspase3; G3 Substrate/Caspase-3: EGY191-2op-LEU2/2op-lacZ/DΔTEF3-Fas-d-G3/7(DEVG)-TA/DΔCYC2-Caspase-3; and (B) S3 Substrate/No Caspase: EGY48-6op-LEU2/2op-lacZ/TEF-Fas-d-S3(DEVD)-TA; S3 Substrate/Caspase-7: EGY48-6op-LEU2/2op-lacZ/TEF-Fas-d-S3(DEVD)-TA/CYC-Caspase-7; G3 Substrate/Caspase-7: EGY48-6op-LEU2/2op-lacZ/TEF-Fas-d-G3 (DEVG)-TA/CYC-Caspase7. These yeast transformants were then subsequently transformed with plasmids expressing a small amount of Caspase-9 (driven from the CYC promoter) or plasmids expressing large amounts of wild-type (WT) or catalyticallydefective (C287→A287) Caspase-9 (driven from the TEF promoter), with or without an active form (gain of function mutant not requiring cytochrome c for activation) of Apaf-1 (driven by TEF-promoter) or the corresponding empty vectors (−) (Haraguchi M, Torii S, Matsuzawa S, et al. J Exp Med 2000; 191: 1709-20).

Note that the large amount of Caspase-9 (expressed from TEF promoter) activated the cleavable S3(DEVD) substrate when co-expressed with pro-Caspase-3 or pro-Caspase-7, but not in their absence, thus constituting a 2-component system. No lacZ reporter gene activity was detected when the non-cleavable substrate (DEVG) was employed (G3). In contrast, expressing a small amount of Caspase-9 (from the CYC promoter) or the catalytically-defective Caspase9 (C287→A287) did not activate Caspase-3 or Caspase-7. However, co-expressing active Apaf-1* with a small amount of pro-Caspase-9 activated the lacZ reporter gene in yeast expressing pro-Caspase-3 or -7 (but not in the absence of these downstream effector Caspases), thus constituting a 3-component system.

### Validation of Yeast-based Assays for Initiator Caspase Activators: Two Component Systems.

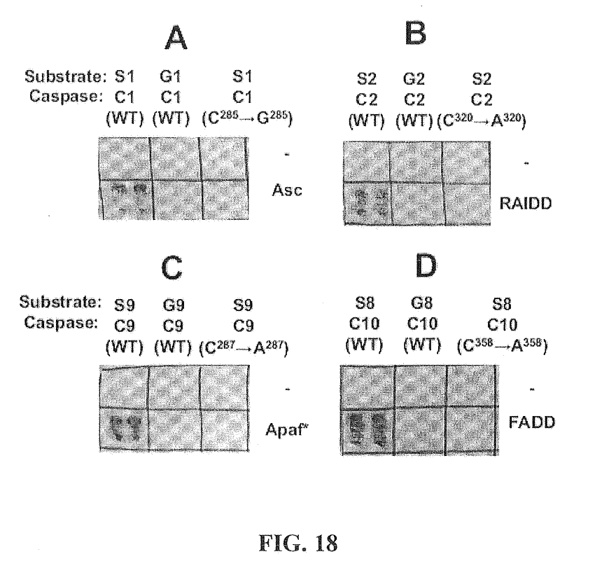

As shown in FIG. 18 consisting of 18A, 18B, 18C and 18D yeast EGY191 strain containing 2op-LEU2/2op-lacZ or EGY48 strain containing 6op-LEU212op-lacZ were employed for developing 2-component assays for activators of upstream initiator Caspases (e.g. Caspases-1, 2, 8, 9, 10). Two independent clones of yeast transformants were plated on leucine-deficient medium containing X-gal. Yeast were transformed with plasmids encoding various membrane tethered transcription factor substrates containing (A) WEHD (“S1”), (B) DEHD (“S2”), (C) LEHD (“S9”) or (D) LETD (“S8”) cleavable linkers or their corresponding noncleavable glycine mutants (“G1”, “G2”, “G9”, “G8”). The yeast were also transformed with plasmids encoding wild-type (WT) or catalytically inactive mutants of proforms various initiator Caspases expressed from relatively weak promoters (e.g., CYC1; ΔTEF3), including (A) pro-Caspase-1, (B) pro-Caspase-2, (C) pro-Caspase-9, and (D) pro-Caspase-10. (Note that the optimal betrapeptide sequences are the same for Caspase-8 and 10). These yeast were transformed with empty vectors (−) or plasmids encoding upstream activators of the Caspases, including (A) Asc, (B) RAIDD, (C) Apaf-1*, and (D) FADD, expressed from strong promoters (either GPD or TEF).

Note that the lacZ reporter gene was activated only when the combination of an initiator Caspase and upstream activator was co-expressed, along with a cleavable substrate.

Transformants. The transformed yeast cell clones are: (A) EGY191-2op-LEU2/2oplacZ/TEF-Fas-d-S1 (WEHD)-TA/ΔTEF1-Caspase1-FLAG (S1, C1(WT)), EGY191-2op-LEU2/2op-lacZ/TEF-Fas-d-G1 (WEHG)-TA/ΔTEF1-Caspase1-FLAG (G1, C1(WT)), or EGY191-2op-LEU2/2op-lacZ/TEF-Fas-d-S1(WEHD)-TA/ΔTEF1-Caspase1 (C285→G285)-FLAG, (S1, C1(C285→G285)), were transformed with the plasmids encoding the activator Asc, or the empty vector (−); (B) EGY48-6op-LEU2/2op-lacZ/ΔTEF1-Fas-d-S2(DEHD)-TA/ΔGPD1-HA-Caspase2-FLAG(S2, C2(WT)), EGY48-6op-LEU2/2op-lacZ/ΔTEF1-Fasd-G2(DEHG)-TA/ΔGPD1-HA-Caspase2-FLAG (G2, C2(WT)), or EGY48-6op-LEU2/2op-lacZ/ΔTEF1-Fas-d-S2(DEHD)-TA/ΔGPD1-HA-Caspase2 (C320→A320)-FLAG (S2, C2(C320→A320)), were transformed with plasmids encoding the activator RAIDD, or the empty vector (−); (C) EGY48-6op-LEU2/2op-lacZ/TEF-Fas-d-S9(LEHD)-TA/TEF-HACaspase9 (S9, C9(WT)), EGY48-6op-LEU2/2op-lacZ/TEF-Fas-d-G9(LEHG)-TA/TEF-HA-Caspase9 (G9, C9(WT)), or EGY48-6op-LEU2/2op-lacZ/TEF-Fas-d-S9(LEHD)-TA/TEF-HA-Caspase9(C287→A287) (S9, C9(C287→A287)), were transformed with plasmids encoding an active form of Apaf-1 (Apaf*), or the empty vector (−); (D) EGY48-6op-LEU2/2op-lacZ/GPD-Fas-d-S8(LETD)-TA/ADH-Caspase10-FLAG (S8, C10(WT)), EGY48-6op-LEU2/2op-lacZ/GPD-Fas-d-G8(LETG)-TA/ADH-Caspase10-FLAG (G8, C10(WT)), or EGY48-6op-LEU2/2op-lacZ/GPD-Fas-d-S8(LETD)-TA/ADHCaspase10(C358→A358)-FLAG (C10(C358→A358)), were transformed with the plasmids encoding the activator FADD, or the empty vector (−). The large amount of FADD is enough to activate Caspases-10 by itself.

Specificity of Upstream Activators of Initiator Caspases—Tested by 2-component Systems.

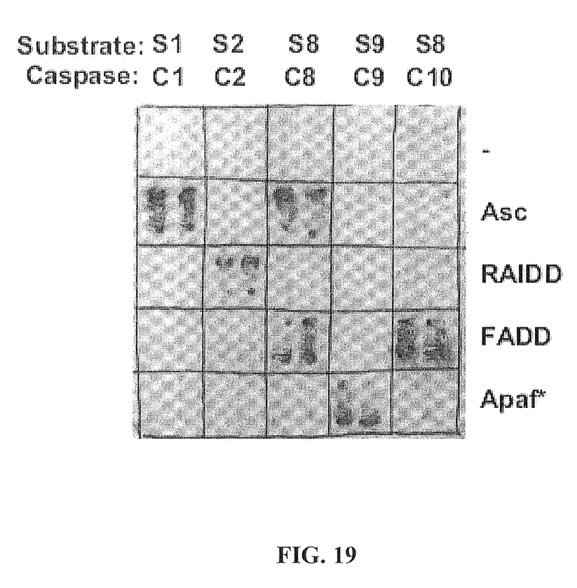

As shown in FIG. 19 yeast transformants were prepared and tested as described in FIGS. 17 and 18, except a matrix of plasmid combinations was prepared to evaluate the specificity of upstream activators. The substrates and Caspases tested are indicated across the top, while the activators are indicated along the side.

Note that results obtained were as predicted, with (1) Apaf-1* activating pro-Caspase-9, but not other initiator Caspases; (2) RAIDD activating pro-Caspase-2, but not other Caspases; (3) FADD activating pro-Caspases-8 and 10, but not other Caspases, and (4) Asc activating pro-Caspase-1 and 8. Note that while Asc contains a CARD that pairs with a complementary CARD in pro-Caspase-1 and would not be necessarily predicted to activate the DED-containing protease Caspase-8, it has previously been reported that Asc is an activator of Caspase-8 (Hasegawa M. et. al. J Biol Chem 280: 15122-30 (2005); Masumoto, J. et. al., Biochem. Biophys. Res. Commun 303: 69-73 (2003).

Transformants: Transformed yeast clones were as follows: S1,C1:EGY191-2op-LEU2/2op-lacZ/TEF-Fas-d-S1 (WEHD)-TA/PΔTEF1-Caspase1-FLAG; S2, C2: EGY48-6op-LEU2/2op-lacZ/PΔTEF1-Fas-d-S2(DEHD)-TA/PΔGPD1-HA-Caspase2-FLAG; S8, C8: EGY48-6op-LEU2/2op-lacZ/GPD-Fas-d-S8/10(LETD)-TA/CYC1-Caspase8-HA; S9, C9: EGY48-6op-LEU2/2op-lacZ/TEF-Fas-d-S9(LEHD)-TA/TEF-HA-Caspase-9; S8, C10: EGY48-6op-LEU2/2op-lacZ/GPD-Fas-d-S8/10(LETD)-TA/ADH-Caspase10-FLAG. These cells were transformed with the plasmids encoding the activators (Asc, RAIDD, FADD, and Apaf*). For controls (−), the “empty” version of the plasmids were introduced.

Validation of 3-component Yeast-based Caspase Assay Reconstituting DISC.

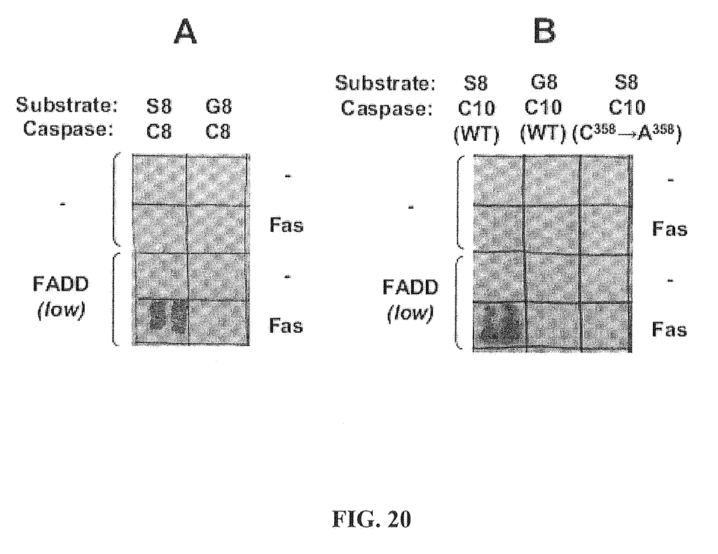

As shown in FIG. 20 consisting of 20A and 20B yeast transformants were prepared to assess the performance of the Fas/FADD/Caspase-8/10 three-component systems. Two independent clones of each transformant were plated on leucine-deficient medium containing X-gal. Substrates included LETD-containing cleavable (S8) and LETG-containing non-cleavable (G8) transcription factors, while Caspase expression plasmids included WT pro-Caspases-8(A) and -10 or a catalytic mutant of Caspase-10. (B) FADD was expressed at low levels from a CYC1 promoter. Note that the lacZ reporter gene was activated only when the combination of Fas, FADD, and either WT pro-Caspase-8 or -10 was co-expressed and only when a cleavable substrate (S8) was employed. (Note that the optimal tetrapeptide cleavage sequence is the same for Caspases-8 and -10).

Transformants: (A) Yeast transformants included EGY48-6op-LEU2/2op-lacZ/TEFFas-d-S8 (LETD)-TA/CYC1-Caspase-8-HA (S8, C8) EGY48-6op-LEU2/2op-lacZ/TEFFas-d-G8(LETG)-TA/CYC1-Caspase8-HA (G8, C8), without (−) or with Fas, and without (−) or with FADD, which were expressed from either ADH and ΔADH or CYC1 promoters, respectively, to achieve high expression of Fas and low expression of FADD. (B) Yeast transformants included: EGY48-6op-LEU2/2op-lacZ/GPD-Fas-d-S8(LETD)-TA/CYC1-Caspase10-FLAG (S8, C10); EGY48-6op-LEU2/2op-lacZ/GPD-Fas-d-G8(LETG)-TA/CYC1-Caspase10-FLAG (G8, C10); and EGY48-6op-LEU2/2op-lacZ/GPD-Fas-d-S8 (LETD)-TA/CYC1-Caspase10(C358→A358)-FLAG (S8, C10(C358→A358)) each without (−) or with Fas, and without (−) or with FADD-expressing vector or the corresponding empty vectors.

Flow Chart for cDNA Library Screening Using 3 Component System—Application to Death Receptor Cloning—Example Screening Strategy #1.

FIG. 21 demonstrates an exemplary screening screening strategy is essentially the same as outlined in FIGS. 6 and 7. The yeast transformant EGY48-6op-LEU2/2op-lacZ/TEF-Fas-d-S8(LETD)-TA/CYC1-Caspase8-HA/ΔGDH1 -FADD was transformed with a HepG2 cell cDNA library (oligo dT-primed, PADH promoter). Independent colonies (1.7×106) appeared on growth plates within 48 hours. Cells were collected, pooled, and a portion (2.4×107) was seeded on the leucine-deficient selection plates containing X-gal. Blue-colored colonies (n=150) appeared within a week, 13 of which encoded DR4, (#XA514), and 15 clones encoded DR5 variant 2 (#ZA512), while the rest were false positives. Screening another cDNA library from HepG2 cells (random-primed, PADH promoter) yielded eight DR5 (#XD108) clones.

Flow Chart for cDNA Library Screening Using 3 Component System to Clone Death Receptors—Example Screening Strategy #2.

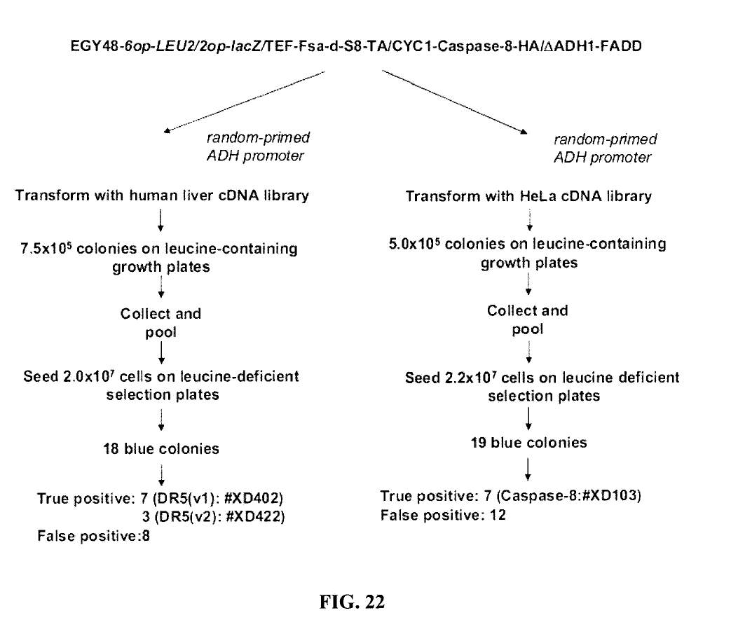

FIG. 22 demonstrates an exemplary screening he screening strategy is essentially the same as outlined in FIGS. 6 and 7. The yeast transformant EGY48-6op-LEU2/2op-lacZ/TEF-Fas-d-S8(LETD)-TA/CYC1-Caspase8-HA/ΔPDH1-FADD was transformed with a human liver cDNA library (random-primed, PADH promoter) and a HeLa cell cDNA library (randomprimed, PADH promoter).

Flow Chart for cDNA Library Screening Using 3-component System to to Clone Death Receptors—Example Screening Strategy #3.

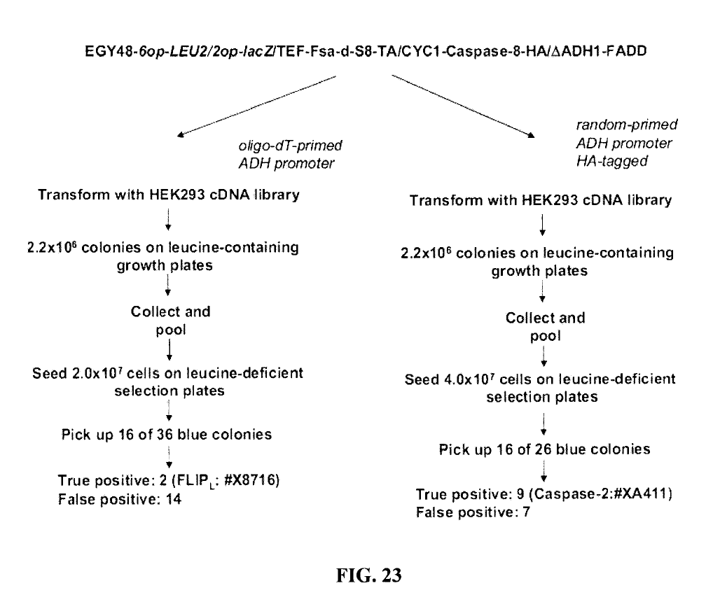

FIG. 23 demonstrates an exemplary screening is essentially the same as outlined in FIGS. 6 and 7. The yeast transformant EGY48-6op-LEU2/2op-lacZ/TEFFas-d-S8(LETD)-TA/CYC1-Caspase8-HA/ΔPDH1-FADD was transformed with a HEK293T cell cDNA library (oligo-dT-primed, PADH promoter) and a HEK293T cell cDNA library (random-primed, PADH promoter, HA-tagged).

Schematic Representation of 3-component System Used for Cloning Adapter Protein that Links Fas to Pro-Caspase-10.

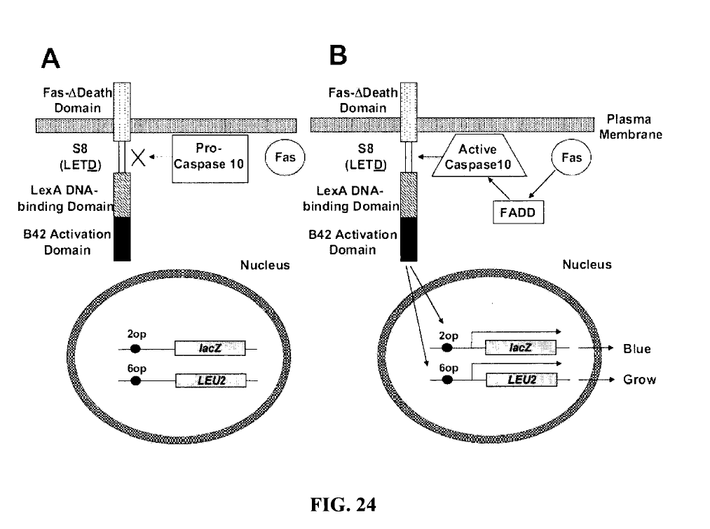

FIG. 24 consisting of 24A and 24B represents a 3-component system for cloning an adapter protein that links Fas to pro-Caspase-10. (A) The zymogen pro-Caspase-10 was expressed (from CYC1-promoter) with substrate containing the LETD-containing cleavage element S8 (from plasmid p413-TEF-Fas-d-S8-TA/CYC1-Caspase-10-FLAG) in the yeast EGY48 expressing 6op-LEU2. Fas was also expressed without activating the Caspase-8 from plasmid p426-2op-lacZ/ADH-Fas. (B) Addition of FADD activates Caspase-8, releasing the transcription factor.

### Validation of Adapter Protein Cloning System for Fas/FADD/Caspase-8/10: Reconstituted DISC.

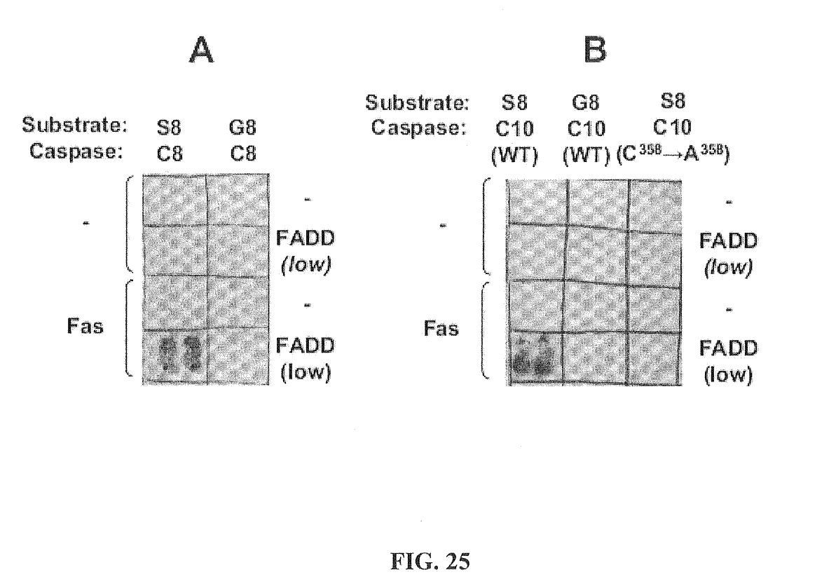

As shown in FIG. 25 consisting of 25A and 25B yeast cell transformants were prepared to assess the performance of the Fas/FADD/Caspase-8 or Fas/FADD/Caspase-10 three-component systems. Two independent clones of each transformant were plated on leucinedeficient medium containing X-gal and grown for 4 days. Substrates included LETD17 containing cleavable (S8) and LETG-containing non-cleavable (G8) transcription factors, while Caspase expression plasmids included wild-type (WT) pro-Caspases-8 (A) and -10 or a catalytic mutant of Caspase-10 (C358→A358) (B). Note that the lacZ reporter gene was activated only when the combination of Fas, FADD, and either WT pro-Caspase-8 or -10 was co-expressed and only when a cleavable substrate (S8) was employed.

Transformants: (A) Yeast cell transformants included: EGY48-6op-LEU2/2op-lacZ/TEFFas-d-S8 (LETD)-TA/CYC1-Caspase8-HA (S8, C8) and EGY48-6op-LEU2/2oplacZ/TEF-Fas-d-G8 (LETG)-TA/CYC1-Caspase8-HA (G8, C8), with empty vector (−) or with plasmids encoding FADD (expressed from CYC1 promoter) or Fas (expressed from ADH promoter). (B) Yeast cell transformants included EGY48-6op-LEU2/2oplacZ/GPD-Fas-d-S8(LETD)-TA/CYC1-Caspase10-FLAG (S8, C10), EGY48-6op-LEU2/2op-lacZ/GPD-Fas-d-G8 (LETG)-TA/CYC1-Caspase10-FLAG (G8, C10), and EGY48-6op-LEU2/2op-lacZ/GPD-Fas-d-S8 (LETD)-TA/CYC1-Caspase10(C358→A358)-FLAG (S8, C10(C358→A358)), each with empty vector (−) or with plasmids encoding FADD or Fas as above.

Flow Chart for cDNA Library Screening Using 3 Component System to Clone Adapters.

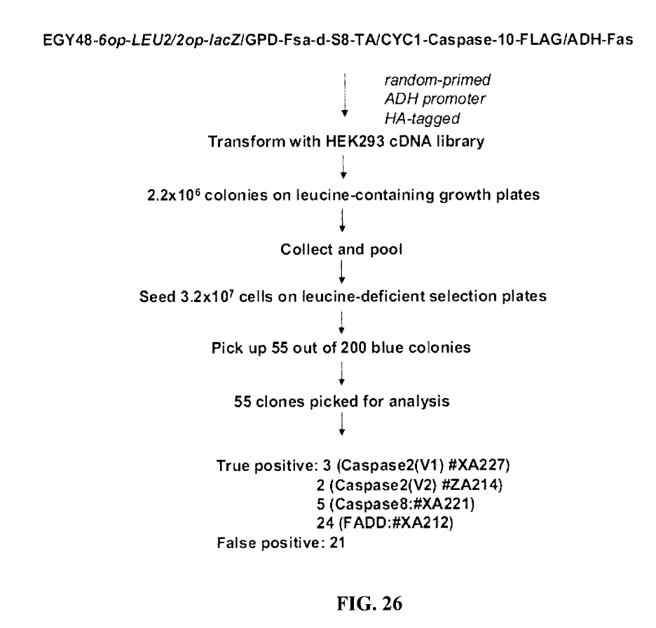

As shown in FIG. 26 the yeast transformant EGY48-6op-LEU2/2op-lacZ/GPD-Fas-d-S 8(LETD)-TA/ADH-Fas/CYC1-Caspase10-FLAG was transformed with a HEK293 cell cDNA library (random-primed, PADH promoter, HA-tagged). Independent colonies (˜2.2×106) appeared on growth plates within 48 hours. Cells were collected, pooled, and a portion (3.2×107) was seeded on leucine-deficient selection plates containing X-gal. Blue-colored colonies (n=200) appeared within a week, five of which encoded fulllength Caspase-2 (#XA227), five encoded a fragment of Caspase-2 (#ZA214), five encoded a fragment of Caspase-8 (#XA221), and 24 encoded full-length FADD (#XA212), while the rest were apparent false positives.

Examples of cDNA Cloning Results.

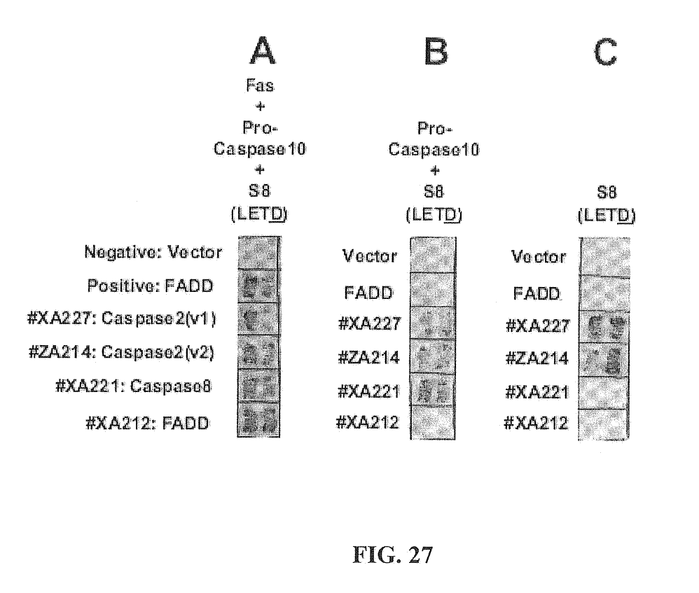

As shown in FIG. 27 consisting of 27A, 27B and 27C the clones that activated the reporter genes were characterized by recovery of cDNA library plasmids and retransformation into yeast expressing Fas and pro-Caspase-10(A), pro-Caspase-10 without Fas (B), or neither (C). Among the positive clones were a full-length Caspase-2 (#XA227), a fragment of Caspase-2 (#ZA214, V130-L312), a fragment of Caspase-8 (#XA221, M1-K438), and full-length FADD (#XA212). Assays were performed in duplicate, growing cells for 4 days on plates.

Optimization of Signal:noise Ratio in Microtiter Plates: Cell Density.

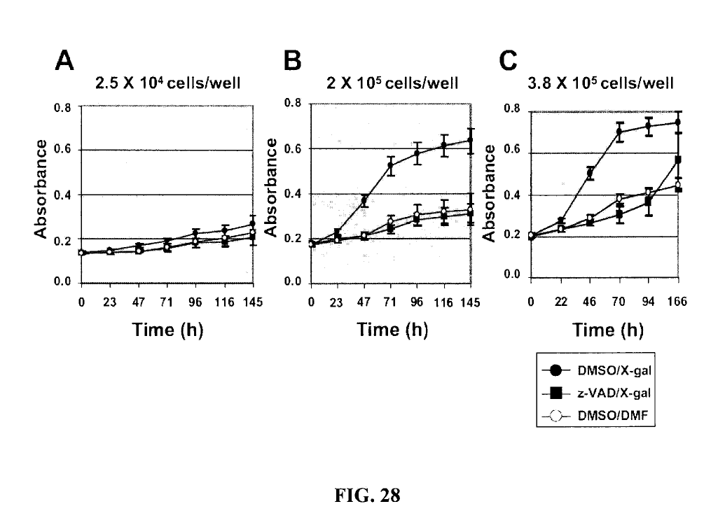

As shown in FIG. 28 consisting of 28A, 28B, and 28D demonstrated that EGY48 cells containing the 3-component Fas/FADD/Caspase-8 system and Caspase-8-cleavable reporter were seeded at 2.35×104/well (A), 2×105/well (B), or 3.76×105/well (C) in 384 well plates to compare cell densities. Cells were cultured at 30° C. The activity of β-galactosidase was measured after 72 hrs at various times after initiating cultures (mean+std dev; n=3), for cells grown without (white circles) or with (black circles and black squares) X-gal and grown in the absence (circles) or presence (black squares) of 100 μM zVAD-fmk Caspase inhibitor.

Optimization of Signal:noise Ratio in Microtiter Plates: Time and X-gal Concentration.

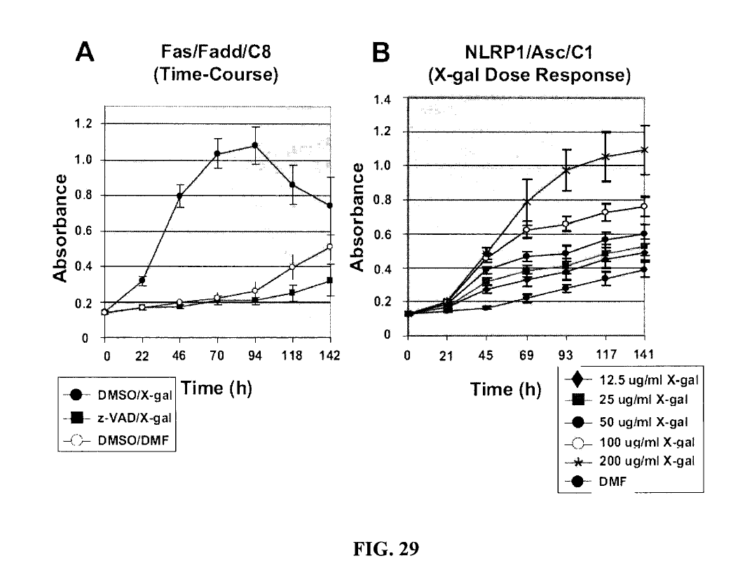

FIG. 29 consisting of 29A, and 29B demonstrates in (A) EGY48 cells containing the 3-component Fas/FADD/Caspase-8 system and Caspase-8-cleavable reporter were grown at 2×105 cells/well in 384 well plates, comparing β-galactosidase activity at various times (mean±std dev; n=3), for cells grown in the absence (black squares) or presence (circles) of X-gal and in the absence (circles) or presence (squares) of 100 μM zVAD-fmk Caspase inhibitor. (B) EGY48 cells containing the 3-component NLRP1ΔLRR/Asc/Caspase-1 system and Caspase-1-cleavable reporter were grown at 2×105 cells/well in 384 well plates, comparing X-gal concentrations. The activity of β-galactosidase was measured after 72 hrs culture (mean±std dev; n=3) for cells grown in the absence (red circles) or presence (all others) X-gal. Various concentration of X-gal were compared, where 1×=50 ug/mL final concentration.

### Plasmids for Co-expression of Caspases and Substrate Cleavable Transcription Factors in Yeast.

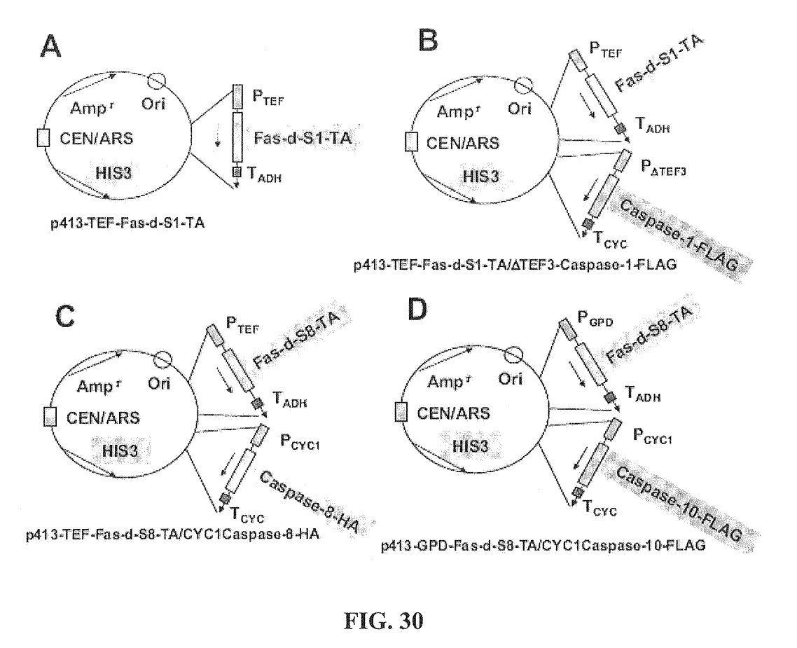

As shown in FIG. 30 consisting of 30A, 30B, 30C and 30D the plasmid p413 was used as the backbone for these constructions, containing CEN/ARS centromeric origin for low-copy episomal replication in yeast (S. cerevisiae) and HIS3 gene for selection in his3 yeast strains. (A) The plasmid p413-TEF-Fas-d-S1-TA/ΔTEF3-Caspase-1-FLAG, contains two additional transcriptional units, where expression of the Fas-LexA/B42 membrane tethered transcription factor with WEHD linker (Caspase-1/4/5 cleavable) is driven by the TEF promoter and expression of pro-Caspase-1 with C-terminal FLAG tag is driven by an attenuated TEF3 promoter (ΔTEF3). (B) The plasmid p413-TEF-Fas-d-S8-TA/CYC1-Caspase-8-HA contains two additional transcriptional units, where expression of the Fas-LexA/1B42 membrane tethered transcription factor with LETD linker (Caspase-8/10 cleavable) is driven by the TEF promoter and expression of pro-Caspase-8 with C terminal HA tag is driven by CYC1 promoter. (C) The plasmid p413-GDP-Fas-dS8-TA/CYC1-Caspase-10-FLAG, similarly contains two additional transcriptional units, where expression of the same Caspase-8/10 cleavable Fas-LexA/B42 substrate as above is driven by GDP promoter and where expression of pro-Caspase-8 with C-terminal FLAG tag is driven by CYC1 promoter. Transcriptional termination elements from the CYC1 and ADH genes were employed as illustrated.

Plasmids for Expression of Upstream Activators of Caspases and lacZ Reporter Gene in Yeast.

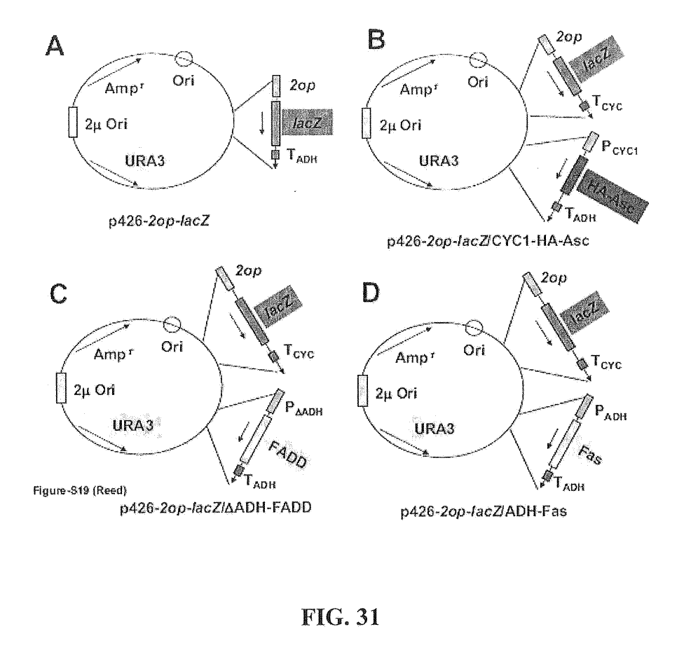

As shown in FIG. 31 consisting of 31A, 31B, 31C and 31D the plasmid p426 was used as the backbone for these constructions, containing 2μ plasmid origin for high-copy episomal replication in yeast and URA3 gene for selection in ura3 yeast strains. (A) The plasmid p426-2op-lacZ contains a lacZ gene driven by a minimal promoter containing two tandem copies of binding sites for the LexA transcription factor, followed by ADH gene termination element. (B) Plasmid p426-2op-lacZ/CYC1-HA-Asc contains an additional transcriptional unit, where expression of human cDNA encoding N-terminal HA-tagged Asc protein is driven by a CYC1 promoter, and followed by ADH gene termination element. (C) In plasmid p426-2op-lacZ/CYC1-FADD, expression of a human FADD cDNA is driven by CYC1 promoter and followed by ADH termination element. (D) Plasmid p426-2op-lacZ/ADH-Fas contains a human Fas(CD95) cDNA driven by ADH promoter, and followed by CYC1 gene termination element.

Yeast Expression Plasmids for Functional Screening of cDNA Libraries and Expression of Upstream Activators of Caspases.

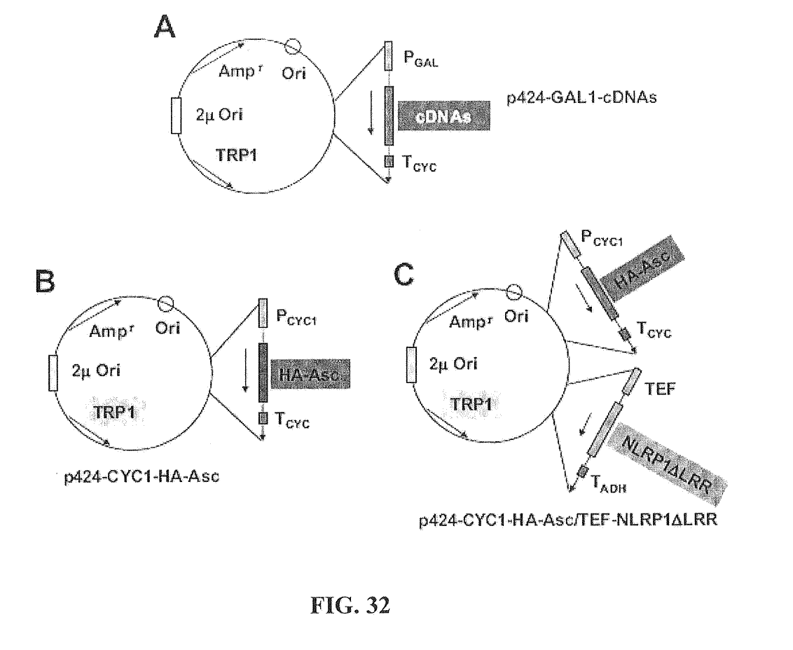

As shown in FIG. 32 consisting of 32A, 32B, and 32C the plasmid p424 was used as the backbone for these constructions, containing 2μ plasmid origin for high-copy episomal replication in trp1 yeast and TRP1 gene for selection in ΔTRP1 yeast strains (S. cerevisiae). (A) The plasmid p426-GAL1-cDNAs contains human cDNAs directionally cloned downstream of a GAL1 promoter, followed by CYC1 gene termination element. (B) Plasmid p424-CYC1-HA-Asc contains a human cDNA encoding N-terminal HA-tagged Asc protein cloned downstream of a CYC1 promoter and followed by CYC1 gene termination element. (C) In plasmid p424-CYC1-HAAsc/TEF-NLRP1ΔLRR, an additional transcriptional unit was added to p424-CYC1-HAAsc above, where expression of a human cDNA encoding a gain-of-function NLRP1 mutant lacking LRRs (Faustin, B, et al. Molecular Cell 25; 713, 2007) is driven by a TEF promoter and followed by ADH gene termination element.

### Experimental Designs & Methods Used

Chemical Library Screens of Multi-component Yeast-based Protease Assay Systems were Performed to Define Hit-rates and Test Reliability.

Chemical library screens to validate the performance of the assays were completed. The screens were conducted such that all assay components were added in automated fashion using integrated robotic liquid handling systems, moving the plates initially into carousels that hold 180 plates at room temperature, and then manually applying seals (breathable sealing film from Axygen Scientific) to reduce evaporation, and moving the bar-coded plates to a 30° C. incubator for the required time (generally culturing for 2-4 days or less). Each assay plate contained a row of positive (min) and a row of negative (max) controls that did not receive compounds but that received DMSO in volumes equivalent to the amount of DMSO in which compounds will be supplied. At the conclusion of the 72 hrs incubation, plates were robotically delivered to one of the integrated multi-purpose plate readers for reading at OD620 nm. The programmable robotic workstations sequenced the additions of reagents, minimizing variations in incubation times. Data from primary screens were uploaded directly from plate readers into computers with customized Microsoft excel software, set up to calculate Z′ factor for each plate, and with hit determinations set at 50% of the mean value for the negative control values.

A screening of a LOPAC was performed at several different concentrations (typically 20, 10, and 5 μM) to compare the hit rates, and to empirically determine an acceptable concentration for conducting large-scale library screens. The empirically adjusted compound screening concentrations allowed for the employment of the highest screening concentration tolerable without causing artifacts or non-specific effects. A hit rate of 0.1-0.5% was achieved which was consistent with the general expectations for the screening results. Before undertaking this study, it was empirically determined what the effects of DMSO on assay performance would be, through pilot experiments where increasing concentrations of DMSO (from 1-10% volume) were added to the positive and negative controls and the impact on assay signal and stability was determined. Second, a progression was made to larger libraries (eg., 50K Chembridge library), it was determined which factors improve the stability of the positive and negative controls from plate to plate, assessing the assay performance as time is extended from minutes to hours, and calculating Z′ for each plate as the quality of the assay's performance is assessed in true screening mode.

For HTS assays the β-galactosidase produced by yeast carrying the caspase-cleavable reporter proteins was measured, by assaying the calorimetric product (OD620 nm) derived from X-gal substrate in 384 well plates, as a measure of the lac Z reporter gene activity.

In summary disclosed are HTS systems for intracellular proteases, using Caspases as a prototype. For this purpose, yeast-based cellular systems that permit facile expression of proteases and protease-activating proteins in combinations that reconstitute entire mammalian pathways in these simple eukaryotes were devised. Among the assay methods integrated into the yeast system are cleavable reporter gene activators, in which protease-mediated cleavage activates a transcription factor. In summary we disclose:

1. Multi-component systems that reconstitute mammalian protease activation pathways in yeast.
2. Optimized systems with adjusted expression levels, reporter gene sensitivity, and other parameters to achieve satisfactory signal:noise results and mechanisms for optimizing future systems.
3. Defined the key variables that require optimization for achieving HTS quality assay performance.
4. Performed chemical library screens of multi-component yeast-based protease assay systems to define hit-rates and test reliability.

### Plasmid Constructions.

### Vectors Encoding Caspase Cleavable Transcription Factors:

The reporter, Fas-d-S1-TA for Caspase-1 and related proteases (called “S1”), was generated by using PCR and standard recombinant DNA techniques. This protein consists of, from N to C termini, amino acids M1-L224 of a type 1 transmembrane protein, human Fas 34 in which Fas is devoid of the cytosolic death domain (Fas-d), a linker containing the sequence GWEHDG between a Xhol and EagI site, and finally a transcriptional activator (TA) containing the LexA DNA binding domain and the B42 activation domain, taken from plasmids pRS305(Δwbpl-Cub-PLV) 35 and pJG4-5 (Invitrogen) with PCR. Other reporters were made by substituting the linker with oligonucleotides designed to encode in-frame the sequences GWEHGG (“G1”), GDEHDG (“S2”), GDEHGG (“G2”), GDEVDG (“S3”), GDEVGG (“G3”), GTEVDG (“S6”), GTEVGG (“G6”), GLETDG (“S8”), GLETGG (“G8”), GLEHDG (“S9”), or GLEHGG (“G9”), after digestion with Xhol and EagI.

### Plasmids for Expressing Caspases and Their Upstream Activators:

Plasmids for expression of Caspases in yeast were derived from pRS series vectors 36-38 and Pesc series vectors (Stratagene [Agilent]). Expression levels were adjusted by using constitutive promoters of different strengths (CYC1, ADH, TEF, and GPD) 39 or using the inducible GAL1 promoter, in conjunction with different strength reporter genes carrying variable numbers of lexA operators 6, and using plasmids with different replication origins for low or high copy number replication (2u and CEN/ARS). To further control expression levels, several attenuated forms of the promoters were made by PCR-assisted deletional mutagenesis. Using a standard indicator gene, the approximate relative strength of the promoters was determined to be: ΔCYC4<ΔCYC2<CYC1<ΔGPD2<ΔGPD1<ΔADH1<ADH<ΔTEF3<ΔTEF2<ΔTEF1<TEF. However, the relative locations of the promoters in complex plasmids somewhat affects their strength, especially when two or three genes are contained in one plasmid. In this regard, two or three genes within one plasmid were sometimes co-expressed by placing the genes flanked by the above promoters and inserting transcription termination elements between them, because the selectable marker genes available are limited (URA3, HIS3, TRP1, and LEU2). In addition, many of the expressed Caspases and upstream activators were cloned with N-terminal HA or C-terminal HA or FLAG epitope tags for convenience of detection of the protein products by immunoblotting. Examples of complex plasmids are (a) p426-2op-lacZ/CYC1-FADD, which consists of 2μ origin, URA3 marker, lacZ gene under the control of two lexA operators, and a FADD gene with expression driven by a short form of the ADH promoter (ΔADH1), as in FIG. 31C, and (b) p413-TEF-Fas-d-S8-TA/CYC1-Caspase8-HA, which consists of CEN/ARS origin, HIS3 marker, Fas-d-S8-TA substrate driven by TEF promoter, and a C-terminally HA-tagged pro-Caspase-8 cDNA under control of the CYC1 promoter, as in FIG. 30C.

### Complex Plasmids Constructions.

Diagrams of complex plasmids containing 3 or more genes are provided in FIGS. 30-32. The cDNAs encoding human Caspases, Adapter proteins, Death Receptors, and NLRs, in wild-type and mutant versions have been described previously J- M. Bruey, et al., Nature Cell Biology 2, 645 (2000). M. Krajewska, et al., Exp Neurol 189 (2), 261 (2004). H. Marusawa, et al., EMBO J 22, 2729 (2003). T. Miyazaki, et al., Nature Immunol 3, 4 (2002). A. D. Schimmer, et al., Cancer Cell 5, 25 (2004).

Construction of Expression cDNA Libraries.

Oligo(dT)-primed or random heptamerprimed cDNA libraries were made in modified p424-GAL1, p424-GAL1-HA, p424-ADH, or p424-ADH-HA plasmids (carrying a TRP1 marker) using mRNAs derived from HEK 293 cells, HepG2 cells, HeLa cells, human liver, or human placenta, as in FIG. 32A. The mRNAs of HepG2 cells, HeLa cells, human liver, and human placenta were purchased from Ambion. The p424-GAL1 was modified to create 5′XhoI and 3′NotI sites downstream of a GAL1 promoter for insertion of the cDNAs. The p424-GAL1-HA has a HA-tag between the GAL1 promoter and the 5′Xhol site. The p424-ADH and p424-ADH-HA plasmids were modified with 5′XhoI and 3′SfiI sites downstream of the ADH promoter. To construct directional libraries, the first-strand cDNA syntheses were initiated with a NotI-oligo(dT) primer adaptor or a NotI-random hexamer primer adaptor for the p424-GAL1 and p424-GAL1-HA, and with a Sfiloligo(dT) primer adaptor or a SfiI-random hexamer primer adaptor for the p424-ADH and p424-ADH-HA, according to the manufacturer's instructions (Invitrogen). SalI adapters were ligated to the resultant double-strand cDNAs prior to digestion with NotI or SfiI. The cDNAs with SalI-NotI termini or with SalI-SfiI termini were ligated into the plasmid cloning vectors (XhoI-NotI-cut or XhoI-SfiI-cut, respectively). The number of initial transformants ranged from 1.3×106 to 1.3×107. A human B cell cDNA library was purchased from ATCC. The oligo(dT)-primed cDNAs were inserted into the XhoI cloning site, downstream of a GAL1 promoter.

### Reporter Gene Assays.

Plasmids were introduced into yeast by lithium acetate transformation. The yeast strain EGY48, which carries 6 lexA operators upstream of LEU2 gene (6op-LEU2), was transformed with pJK103 6, which carries two lexA operators upstream of lacZ gene (p426-2op-lacZ), and subsequently with reporter plasmids (p413-TEF-Fas-d-S1-TA, p413-TEF-Fas-d-G1-TA, p413-TEF-Fas-d-S2-TA, p413-TEF-Fas-d-S3-TA, p413-TEF-Fas-d-S6-TA, p413-TEF-Fas-d-S8-TA, or p413-TEF-Fas-d-S9-TA). Caspase (all full-length zymogen proforms) expression plasmids (p424-ADH-Caspasel-FLAG, p424-ADH-HA-Caspase2, p424-ADH-Caspase3, p424-TEF-Caspase4, p424-ADH-Caspase5, p424-TEF-HA-Caspase6, p424-ADH-Caspase7, and p424-ADH-HA-Caspase9-FLAG) or empty vector (p424-ADH), were introduced into these backgrounds. As for assays with Caspase-8 and -10, small amounts of pro-Caspase-8 and -10 were expressed with a large amount of FADD by transforming yeast with the dual gene plasmids p424-CYC1-Caspase8-HA/TEF-HA-FADD and p424-CYC1-Caspase10-FLAG/TEF-HA-FADD, because expression of large amounts of human Caspase-8 or Caspase-10 significantly inhibited the cell growth. The transformants (two independent colonies for each transformation) were streaked on growth plates (minimum synthetic dropout (SD) medium containing 2% glucose and 50 μg/ml leucine) or on selection plates (SD medium containing 1% galactose, 0.2% raffinose, BU salts, and 80 μg/ml X-gal). Yeast growth and blue color development were monitored for four to six days at 30° C.

Reporter Gene Assays in Liquid Media Using 384-well Plates.

Assays were performed in a total volume of 40 ηl in triplicate. First, 20 μl of liquid selection media containing X-gal and a series of concentrations of reagents such as z-VAD was dispensed into each well of 384-well plates. Next, confluent yeast cells expressing various Caspases and cleavable substrates were collected, washed with water, and suspended in selection media of the same volume as the culture media. The yeast suspension was diluted to 1:5-10 (v:v) with the selection media, and 20 μl aliquots were added to the 384-well plates. Absorbance at 620 nm was measured 2-3 days after culture at 30° C.

### HTS Assays.

For the Fas/FADD/Caspase-8 and the NLRP1/Asc/Caspase-1 assays, EGY48 yeast containing the desired plasmids were streaked onto SD plates (6.8 g Yeast Nitrogen Base w/o amino acids, 20 mg arginine, 50 mg threonine, 30 mg isoleucine, 60 mg phenylalanine, 20 mg valine) containing agar (1.7%), supplemented with 2% α-D-glucose and 50 μg/mL leucine. The plates were incubated at 30° C. for 48 hrs and a colony was picked and transferred to a 14 ml polypropylene tube containing 2 ml of SD media broth supplemented with α-D-glucose and leucine as above and grown at 30° C. for 16-24 hrs with shaking. Then, 1 ml of the overnight culture was transferred into 20 mls of growth media in a 500 ml flask and shaken at 30° C. for 16-24 hrs. The cells were collected by centrifugation at 1000×g for 5 minutes at room temperature, and washed with 20 mls of sterile water, then resuspended in 20 mls of SD broth supplemented with 1% galactose and 0.2% raffinose. The HTS Assay was performed at a final compound concentration of 10 μM (1% DMSO), with cell densities of 2×105 cells/ml (2×105 cells) in a volume of 40 μl. The assay was assembled by the addition of 4 μl of ≈100 μM compounds (final concentration of 10 μM) in 10% DMSO (1% final DMSO) to clear polystyrene 384-well microplates using a Beckman-Coulter Biomek FX, then 18 μl of cell suspension (1.1×107 cells/ml) (2×105 cells/well) and 18 μl of X-gal suspended in Selection Media (for a final concentration of 400 μg/mL) were added to the wells using a Matrix WellMate bulk reagents dispenser. Remaining solutions were added by using the WellMate from Matrix Technologies Corp. Controls were included with each plate, corresponding to cells treated with DMSO (without compounds) and cells cultured with and without X-gal. The plates were sealed with breathable sealing film (from Axygen Scientific) to reduce evaporation and transferred to 30° C. incubators. After 48 hours, the plates were brought to room temperature, the breathable film removed and replaced with the transparent polyester tape seal, and the plates were mixed, pelleted briefly and read using a Beckman DTX 880, recording absorbance at 620 nm.

### Yeast-based Counter-screen Assays.

EGY48 yeast inducibly (GAL1 promoter) expressing the LexA/B42 transcription factor or containing the empty vector were used to detect compounds that directly inhibit the lacZ reporter gene or that alter β-galactosidase activity. The plasmids employed were p424-GAL1 and p424-GAL1-TA (transcriptional activator) with transformed cells selected on tryptophan-deficient plates. Yeast expressing Caspase-1 activated by expression of high levels of Asc (ΔTEF3-Caspase-1-FLAG/TEF-HA-Asc) were used to detect compounds that cross-react with hits from the Fas/FADD/Caspase-8 screen. The Asc/Caspase-1 yeast cells were cultured and assayed under identical conditions to those for the primary NLRP1 HTS assay (see above), using cells expressing 6op-LEU2/2op-lacZ reporter genes and TEFFas-d-S1-TA substrate.

### Cloning Systems for Caspase Activators.

To clone cDNAs encoding proteins that activate Caspase-1, the yeast strain EGY191 containing 2op-LEU2/2op-lacZ was transformed with plasmids containing transcriptional units of TEF-Fas-d-S1-TA (substrate) and ΔTEF3-Caspase1-FLAG (pro-Caspase-1), as illustrated in FIG. 30B, and selected on histidine-deficient plates. As controls, cells were subsequently transformed with the Asc-expressing plasmid p424-TEF-HAAsc or empty vector and selected on tryptophan-deficient plates.

To clone cDNAs encoding proteins that activate Caspase-2, the yeast strain EGY48 containing 6op-LEU2/2op-lacZ was transformed with plasmids containing transcriptional units of ΔTEF2-Fs-d-S2-TA (substrate) and ΔGPD1-HA-Caspase2-FLAG (pro-Caspase-2) and selected on histidine-deficient plates. As controls, cells were subsequently transformed with the RAIDD-expressing plasmid p424-TEF-HA-RAIDD or empty vector and selected an tryptophan-deficient plates.

To clone cDNAs encoding proteins that activate Caspase-3, the yeast strain EGY191 containing 2op-LEU2/2op-lacZ was transformed with plasmids containing transcriptional units for ΔTEF3-Fas-d-S3-TA and ΔCYC2-Caspase-3 and selected on histidine-deficient plates. As controls, cells were transformed with the Caspase-9-expressing plasmid p424-ADH-HA-Caspase9-FLAG or empty vector and selected on tryptophan-deficient plates.

To clone cDNAs encoding proteins that activate Caspase-4, the yeast strain EGY48 containing 6op-LEU2/2op-lacZ was transformed with plasmids containing transcriptional units for GPD-Fas-d-S1-TA and ΔTEF2-Caspase4. As controls, cells were transformed with a plasmid expressing high levels of (Caspase-7 (p424-ADHCaspase-7), which results in active Caspase-7 did but does not cut the S1 site.

To clone molecules that activate Caspase-7, the yeast strain EGY48 expressing 6op-LEU2/2op-lacZ/TEF-Fas-d-S3-TA/CYC1-Caspase-7 was created. The small amount of Caspase-7 exists as inactive zymogen, and does not cut the S3 site. As a positive control, pro-Caspase-7 was activated by expressing Caspase-9 at high levels (p424-ADH-HA-Caspase9-FLAG). Active Caspase-9 did not cut the S3 site.

To clone cDNAs encoding proteins that activate Caspase-8, the yeast strain EGY48 containing 6op-LEU2/2op-lacZ was transformed with plasmids containing transcriptional units for GPD-Fas-d-S8-TA and CYC1-Caspase8-HA and selected on histidine-deficient plates. As controls, cells were transformed with FADD-expression plasmid p424-TEF-HA-FADD or empty vector and selected on tryptophan-deficient plates.

To clone cDNAs that activate Caspase-9, the yeast strain EGY48 containing 6op-LEU2/2op-lacZ was transformed with plasmids containing transcriptional units for TEF-Fas-d-S9-TA and TEF-HA-Caspase9, on a low-copy plasmid p413 (CENLARS). The small amount of pro-Caspase-9 exists as inactive zymogen, and does not cut the S9 site. As controls, cells were transformed with the Apaf-1*-expressing plasmid p424-TEF-HA-Apaf* or empty vector and selected on tryptophan-deficient plates.

To clone cDNAs encoding proteins that activate Caspase-10, the yeast strain EGY48 containing 6op-LEU212op-lacZ was transformed with plasmids containing transcriptional units for GPD-Fas-d-S8-TA and ADH-Caspase10-FLAG and selected on histiodine-deficient plates. As controls, cells were transformed with the FADD expressing plasmid p424-TEF-HA-FADD or empty vector and selected on tryptophan deficient plates.

Procedures for cDNA Library Screening.

Screens for cDNAs encoding proteases (Caspases). To screen for cDNAs encoding proteases capable of cleaving the S1 site (WEHD), the yeast strain EGY48 containing 6op-LEU212op-lacZ was transformed with p413-TEF-Fas-d-S1-TA followed selection on histiodine deficient plates, then these cells were subsequently transformed with a HEK293 cDNA library (contains TRP1 marker) or a human placenta cDNA library (contains TRYP1 marker). The transformants were seeded on tryptophan-deficient growth plates (SD medium containing 2% glucose and 50 ug/ml leucine). Independent colonies of 5.1×105 appeared from the HEK293 cDNA library in 48 hours. The colonies were harvested and pooled, and a portion of the recovered cells (3.6×106 cells) was seeded onto leucine-deficient selection plates (SD medium containing 1% galactose, 0.2% raffinose, BU salts, and 80 ug/ml X-gal). Bluecolored colonies appeared within a week and were subjected to plasmid DNA extraction. The extracted plasmid DNAs were introduced into KC8 E. coli cells by electroporation to efficiently recover the cDNAs plasmids. The candidate cDNAs were again introduced into the yeast cells containing 6op-LEU2/2op-lacZ/TEF-Fas-d-S1-TA or 6op-LEU2/2op-lacZ/TEF-Fas-d-G1-TA to confirm whether they cleave S1 specifically.

To screen for cDNAs encoding proteases capable of cleaving the S3 site (DEVD), the yeast strain EGY48 containing 6op-LEU2/2op-lacZ, was transformed with p413-TEF-Fas-d-S3-TA, followed by selection on histiodine-deficient plates, and subsequently transformed with a HEK293 cell cDNA library, then processed as above.

Screening for cDNAs Encoding Proteins That Activate Caspase-8 in a FADD-dependent Manner.

The yeast strain EGY48 (6op-LEU2) was transformed with plasmids p426-2op-lacZ/ΔADH1-FADD and p413-TEF-Fas-d-S8-TA/CYC1-Caspase8, then subsequently transformed with a HepG2 cDNA library, a human liver cDNA library, or a HEK293 cDNA library. The transformants were seeded on growth plates (SD medium containing 2% glucose and 50 ug/ml leucine). Independent colonies of 1.7×106 appeared from the HepG2 library in 48 hours. They were harvested, and pooled, and a portion of the cells (2.4×107 cells) was seeded on selection plates (SD medium containing 1% galactose, 0.2% raffinose, BU salts, and 80 ug/ml X-gal). Blue-colored colonies appeared within a week, were subjected to plasmid DNA extraction. The extracted plasmid DNAs were introduced into KC8 E. coli cells by electroporation to recover the cDNAs plasmids. The candidate cDNAs were again introduced into the yeast cells containing 6op-LEU2/2op-lacZ and plasmids containing transcriptional units for ΔADH1-FADD, TEF-Fas-d-S8-TA, and CYC1-Caspase8; or TEF-Fas-d-S8-TA and CYC1-Caspase8; or TEF-Fas-d-S8-TA to confirm whether they activate Caspase-8 in a FADD-dependent manner.

Screening for cDNA Encoding Adapter Proteins That Link Fas or DR5 to Caspases-8 and -10.

The yeast strain EGY48 (6op-LEU2) was transformed with p426-2op-lacZ/ADHDR5-FLAG and p413-TEF-Fas-d-S8-TA/CYC1-Caspase8, then subsequently transformed with a HeLa cell cDNA library or a HEK293 cDNA library. The transformants were seeded on growth plates (SD medium containing 2% glucose and 50 ug/ml leucine). Independent colonies of 1.0×106 appeared from the HeLa cell cDNA library within 48 hours. They were harvested, pooled, and a portion of the cells (2.4×107 cells) was seeded on selection plates (SD medium containing 1% galactose, 0.2% raffinose, BU salts, and 80 ug/ml X-gal). Blue-colored colonies appeared within a week and were subjected to plasmid DNA extraction. The extracted plasmid DNAs were introduced into KC8 E. coli cells by electroporation to recover the cDNA plasmids. The candidate cDNAs were again introduced into the yeast cells expressing 6op-LEU2/2oplacZ/ADH-DR5-FLAG/TEF-Fas-d-S8-TA/CYC1-Caspase8, 6op-LEU2/2op-lacZ/TEFFas-d-S8-TA/CYC1-Caspase8, or 6op-LEU2/2op-lacZ/TEF-Fas-d-S8-TA to confirm whether they activate Caspase-8 in a DR5-dependent manner.

The yeast strain EGY48 (6op-LEU2) was transformed with p426-2op-lacZ/ADH-Fas and p413-GPD-Fas-d-S8-TA/CYC1-Caspase10, then subsequently transformed with a HEK293 cell cDNA library. The transformants were seeded on growth plates (SD medium containing 2% glucose and 50 ug/ml leucine). Independent colonies of 2.2×106 appeared within 48 hours, and were harvested, and pooled. A portion (3.2×107 cells) of the cells seeded on selection plates (SD medium containing 1% galactose, 0.2% raffinose, BU salts, and 80 ug/ml X-gal). Blue-colored colonies appeared in a week and were subjected to plasmid DNA extraction. The extracted plasmid DNAs were introduced into KC8 E. coli cells by electroporation to recover the cDNA plasmids. The candidate cDNAs were again introduced into the yeast cells expressing 6op-LEU2/2oplacZ/ADH-Fas/GPD-Fas-d-S8-TA/CYC1-Caspase10, 6op-LEU2/2op-lacZ/GPD-Fas-d-S8-TA/CYC1-Caspase10, or 6op-LEU2/2op-lacZ/GPD-Fas-d-S8-TA to confirm whether they activate Caspase-10 in a Fas-dependent manner.

### REFERENCES

Alnemri E S, Livingston D J, Nicholson D W, et al. Human ICE/CED-3 Protease Nomenclature. Cell 1996; 87: 171.

Ashkenazi A, Dixit V M. Death receptors: signaling and modulation. Science 1998; 281: 1305-8.

Baliga B C, Colussi P A, Read S H, Dias M M, Jans D A, Kumar S. Role of prodomain in importin-mediated nuclear localization and activation of caspase-2. J Biol Chem 2003; 278: 4899-905.

Chinnaiyan A, Chaudhary D, O'Rourke K, Koonin E, Dixit V. Role of CED-4 in the activation of CED-3. Nature 1997; 388: 728-9.

Cirman T, Oresic K, Mazovec G D, et al. Selective disruption of lysosomes in HeLa cells triggers apoptosis mediated by cleavage of Bid by multiple papain-like lysosomal cathepsins. J Biol Chem 2004; 279: 3578-87.

Estojak J, Brent R, Golemis E A. Correlation of two-hybrid affinity data with in vitro measurements. Mol Cell Biol 1995; 15: 5820-9.

Faustin B, Lartigue L, Bruey J M, et al. Reconstituted NALP1 inflammasome reveals two-step mechanism of Caspase-1 activation. Molecular Cell 2007; 25(5): 713-24.

Fischer H, Koenig U, Eckhart L, Tschachler E. Human caspase-12 has acquired deleterious mutations. Biochem Biophys Res Comm 2002; 293: 722-6.

Hawkins C J, Wang S L, Hay B A. A cloning method to identify caspases and their regulators in yeast: Identification of Drosophila DIAP1 as an inhibitor of the Drosophila caspase DCP-1. Proc Natl Acad Sci USA 1999; 96: 2885-90.

Hawkins C J, Yoo S J, Peterson E P, Wang S L, Vernooy S Y, Hay B A. The drosophila caspase DRONC cleaves following glutamate or aspartate and is regulated by DIAP1, HID, and GRIM. J Biol Chem 2000; 275: 27084-93.

Hornig N C, Knowles P P, McDonald N Q, Uhlmann F. The dual mechanism of separase regulation by securin. Curr Biol 2002; 12(12): 973-82.

Irmler M, Thome M, Hahne M, et al. Inhibition of death receptor signals by cellular FLIP. Nature 1997; 388: 190-5.

Jin C, Reed J C. Yeast and apoptosis. Nature Rev Mol Cell Biol 2002; 3: 453-9.

Kamada S, Kusano H, Fujita H, et al. A cloning method for caspase substrates that uses the yeast two-hybrid system: Cloning of the antiapoptotic gene gelsolin. Proc Natl Acad Sci USA 1998; 95: 8532-7.

Kang J, Schaber M, Srinivasula S, et al. Cascades of mammalian caspase activation in the yeast Saccharomyces cerevisiae. J Biol Chem 1999; 274: 3189-98.

Kanneganti T D, Ozoren N, Body-Malapel M, et al. Bacterial RNA and small antiviral compounds activate caspase-1 through cryopyrin/Nalp3. Nature 2006; 440(7081): 233-6.

Kluck R M, Bossy-Wetzel E, Green D R, Newmeyer D D. The release of cytochrome c from mitochondria: a primary site for Bcl-2 regulation of apoptosis. Science 1997; 275: 1132-6.

Kuechle M K, Predd H M, Fleckman P, Dale B A, Presland R B. Caspase-14, a keratinocyte specific caspase: mRNA splice variants and expression pattern in embryonic and adult mouse. Cell Death Differ 2001; 8: 868-70.

Li P, Nijhawan D, Budihardjo I, et al. Cytochrome c and dATP-dependent formation of Apaf-1/Caspase-9 complex initiates an apoptotic protease cascade. Cell 1997; 91 :479-89.

Liu X, Kim C N, Yang J, Jemmerson R, Wang X. Induction of apoptotic program in cell-free extracts: requirement for dATP and Cytochrome C. Cell 1996; 86: 147-57.

Lopez-Otin C, Overall C M. Protease degradomics: a new challenge for proteomics. Nat Rev Mol Cell Biol 2002; 3(7): 509-19.

Mariathasan S, Weiss D S, Newton K, et al. Cryopyrin activates the inflammasome in response to toxins and ATP. Nature 2006; 440(7081): 228-32.

Martinon F, Bums K, Tschopp J. The Inflammasome: A molecular platform triggering activation of inflammatory caspases and processing of proIL-b. Mol Cell 2002; 10: 417-26.

Martinon F, Tschopp J. NLRs join TLRs as innate sensors of pathogens. Trends Immunol 2005; 26: 447-54.

McCusker F, Schreuder P A. Comment: Reversal reactions in leprosy and their management. Lepr Rev 1992; 63(2): 184.

Muzio M, Stockwell B R, Stennicke H R, Salvesen G S, Dixit V M. An induced proximity model for caspase-8 activation. J Biol Chem 1998; 273: 2926-30.

Nagao K, Yanagida M. Securin can have a separase cleavage site by substitution mutations in the domain required for stabilization and inhibition of separase. Genes Cells 2006; 11 (3): 247-60.

Nagata S, Golstein P. The Fas death factor. Science 1995; 267(5203): 1449-56.

Paroni G, Henderson C, Schneider C, Brancolini C. Caspase-2 can trigger Cytochrome c release and apoptosis from the nucleus. J Biol Chem 2002; In Press.

Reed J C. Caspases and cytokines: roles in inflammation and autoimmunity. Adv Immunol 1999; 73: 265-99.

Reed J C. Cytochrome C: Can't live with it; Can't live without it. Cell 1997; 91: 559-62.

Ross K E, Cohen-Fix O. Separase: a conserved protease separating more than just sisters. Trends Cell Biol 2002; 12(1): 1-3.

Saleh A, Srinivasula S, Acharya S, Fishel R, Alnemri E. Cytochrome c and dATP-mediated oligomerization of Apaf-1 is a prerequisite for procaspase-9 activation. J Biol Chem 1999; 274: 17941-5.

Saleh M, Mathison J C, Wolinski M K, et al. Enhanced bacterial clearance and sepsis resistance in caspase-12-deficient mice. Nature 2006; 440: 1064-8.

Salvesen G S, Dixit V M. Caspases: Intracellular signaling by proteolysis. Cell 1997; 91: 443-6.

Salvesen G S, Renatus M. Apoptosome: The seven-spoked death machine. Develop Cell 2002; 2: 256-7.

Seshagiri S, Miller L K. Caenorhabditis elegans CED-4 stimulates CED-3 processing and CED-3-induced apoptosis. Curr Biol 1997; 7: 455-60.

Slee E, Harte M, Kluck R, et al. Ordering the cytochrome c-initiated caspase cascade: Hierarchic activation of caspases-2,-3,-6,-7,-8 and -10 in a caspase-9-dependent manner. J Cell Biol 1999; 144: 281-92.

Stehlik C, Reed J C. The PYRIN connection: novel players in innate immunity and inflammation. J Exp Med 2004; 200: 551-8.

Stennicke HR, Jürgensmeier J M, Shin H, et al. Pro-caspase-3 is a major physiologic target of caspase-8. J Biol Chem 1998; 273: 27084-90.

Sullivan M, Hornig N C, Porstmann T, Uhlmann F. Studies on substrate recognition by the budding yeast separase. J Biol Chem 2004; 279(2): 1191-6.

Talanian R, Quinlan C, Trautz S, et al. Substrate specificities of caspase family proteases. J Biol Chem 1997; 272: 9677-82.

Thornberry N A, Lazebnik Y. Caspases: enemies within. Science 1998; 281: 1312-6.

Thornberry N A, Rano T A, Peterson E P, et al. A combinatorial approach defines specificities of members of the caspase family and granzyme B. J Biol Chem 1997; 272: 17907-11.

Timmer J C, Salvesen G S. Caspase substrates. Cell Death Differ 2007; 14(1): 66-72.

Tinel A, Tschopp J. The PIDDosome, a protein complex implicated in activation of caspase-2 in response to genotoxic stress. Science 2004; 304: 843-6.

Tschopp J, Irmler M, Thome M. Inhibition of Fas death signals by FLIPs. Curr Opin Immunol 1998; 10: 552-8.

Viadiu H, Stemmann O, Kirschner M W, Walz T. Domain structure of separase and its binding to securin as determined by E M. Nat Struct Mol Biol 2005; 12(6): 552-3.

Wallach D, Boldin M, Varfolomeev E, Beyaert R, Vandenabeele P, Fiers W. Cell death induction by receptors of the TNF family: towards a molecular understanding. FEBS Lett 1997; 410: 96-106.

Wallach D. Placing death under control. NATURE 1997; 388: 123-6.

Yang J, Liu X, Bhalla K, et al. Prevention of apoptosis by Bcl-2: release of cytochrome c from mitochondria blocked. Science 1997; 275: 1129-32.

Yang X, Chang H, Baltimore D. Essential role of CED-4 oligomerization in CED-3 activation and apoptosis. Science 1998; 281: 1355-7.

Yang X, Chang H Y, Baltimore D. Autoproteolytic activation of pro-caspases by oligomerization. Mol Cell 1998; 1: 319-25.

Yuan J, Shaham S, Ledoux S, Ellis H, Horvitz H. The C. elegans cell death gene ced-3 encodes a protein similar to mammalian interleukin-1 beta-converting enzyme. CELL 1993; 75: 641-52.

Zhang H, Reed J C. Studies of apoptosis proteins in yeast. In: Schwartz L, Ashwell, editors. Methods in Cell Biology. 2nd ed: Academic Press; 2001. p. 453-68.

Zhang J H, Chung T D, Oldenburg K R. A Simple Statistical Parameter for Use in Evaluation and Validation of High Throughput Screening Assays. J Biomol Screen 1999; 4(2): 67-73.

Zou H, Henzel W J, Liu X, Lutschg A, Wang X. Apaf-1, a human protein homologous to C. elegans CED-4, participates in cytochrome c-dependent activation of caspase-3. Cell 1997; 90: 405-13.

Zou H, Li Y, Liu X, Wang X. An APAF-1 cytochrome c multimeric complex is a functional apoptosome that activates procaspase-9. J Biol Chem 1999; 274: 11549-56.

All references cited herein are hereby incorporated by reference in their entireties, whether previously specifically incorporated or not. As used herein, the terms “a”, “an”, and “any” are each intended to include both the singular and plural forms.

Having now fully described the disclosed subject matter, it will be appreciated by those skilled in the art that the same can be performed within a wide range of equivalent parameters, concentrations, and conditions without departing from the spirit and scope of the disclosure and without undue experimentation. While this disclosure has been described in connection with specific embodiments thereof, it will be understood that it is capable of further modifications. This application is intended to cover any variations, uses, or adaptations of the subject matter following, in general, the principles of the disclosure and including such departures from the disclosure as come within known or customary practice within the art to which the subject matter pertains and as may be applied to the essential features hereinbefore set forth.

Although the invention has been described with reference to the above example, it will be understood that modifications and variations are encompassed within the spirit and scope of the invention. Accordingly, the invention is limited only by the following claims.

What is claimed is:

1. A reporter gene system for monitoring protease activity in living yeast comprising:
singly or in concert a exogenous protease with or without a exogenous activator of said protease;
wherein said exogenous protease cleaves a protein;
wherein said protein is a transcription factor;
wherein said transcription factor induces the expression of one or more reporter genes;
wherein the presence of one or more reporter genes facilitates the ability of said yeast to grow in a leucine deficient media;
wherein a color culture is generated by the cleavage of a substrate by a gene product; and
wherein said color culture assists with determining the protease activity of said exogenous protease.

2. The reporter gene system of 1 claim wherein said protease is caspase.

3. The reporter gene system of claim 2 wherein said protein is a chimeric protein.

4. The reporter gene system of claim 3 wherein said one or more reporter genes are LacZ and LEU2.

5. The reporter gene system of claim 4 wherein said one or more reporter genes is LEU2.

6. The reporter gene system of claim 5 wherein said color culture is blue.

7. The reporter gene system of claim 6 wherein said blue color culture is generated by the cleavage of X-gal by the LacZ gene product, β-galactosidase.

8. The reporter gene system of claim 7 wherein the intensity of said blue color is measured by colorimetry and quantified with OD units.

9. The reporter gene system of claim 8 wherein said OD units are directly proportional to the activity of the caspase in the system.

10. The reporter gene system of claim 9 wherein said OD units provide a read out value which may be further analyzed to qualitatively or quantitatively determine caspase activity in the system.

11. A reporter gene system for monitoring protease activity in living yeast comprising:
singly or in concert a exogenous recombinant protease and/or a exogenous upstream activator of said protease;
wherein said recombinant protease cleaves a chimeric protein;
wherein said chimeric protein is a transcription factor;
wherein said transcription factor induces the expression of the LacZ and LEU2 genes;
wherein the presence of said LEU2 gene facilitates the ability of said yeast to grow in a leucine deficient media;
wherein a blue color culture is generated by the cleavage of X-gal or other substrates by the LacZ gene product, β-galactosidase;
wherein said blue color culture assists with determining the protease activity of said exogenous recombinant protease and/or said exogenous upstream activator of said protease.

12. The reporter gene system of claim 11 wherein said protease activity is caspase activity; wherein said exogenous recombinant protease is a exogenous recombinant caspase and said exogenous upstream activator of said protease is a exogenous upstream activator of caspase when present.

13. The reporter gene system of claim 12 wherein;
the intensity of said blue color is measured by colorimetry and quantified with OD units;
wherein said OD units are directly proportional to the activity of the caspase in the system;
wherein said OD units provide a read out value which may be further analyzed to qualitatively or quantitatively determine caspase activity in the system.

14. A method of generating and using a genetic system for monitoring from exogenous caspase activation pathways in yeast comprising:
singly or in concert a exogenous recombinant caspase with or without an exogenous upstream activator of caspase;
wherein said recombinant caspase cleaves a chimeric protein;
wherein said chimeric protein is a transcription factor;
wherein said transcription factor induces the expression of the LacZ and LEU2 genes;
wherein the presence of said LEU2 gene facilitates the ability of said yeast to grow in a leucine deficient media;
wherein a color culture is generated by the cleavage of X-gal or other substrates by the LacZ gene product, β-galactosidase;
wherein the intensity of said blue color is measured by colorimetry and quantified with OD units; and
wherein said OD units are directly proportional to the activity of the caspase in the system.

15. The method of claim 14 where the yeast is Saccharomyces cerevisiae.

16. A method of generating and using a genetic system for monitoring from exogenous caspase activation pathways in yeast comprising:
a single-component system wherein said exogenous caspase is stably over-expressed and autoactivated in a yeast strain containing a transmembrane cleavable transcription factor;
wherein said transmembrane cleavable transcription factor is expressed such that a the majority of the cytosolic domain of said transmembrane receptor is replaced by a chimeric transcription factor;
wherein said chimeric transcription factor comprises the DNA-binding domain of LexA and the transactivation domain of B42 and is described as LexA-B42 chimeric transcription factor;
wherein between the Fas and the LexA-B42 domains are one or more tetrapeptide sequences known to be recognized and cleaved by various members of the Caspase family;
wherein an appropriately matched set of the appropriate caspase to an appropriate chimera results in a cleavable transcription factor
wherein said transcription factor induces the expression of a reporter gene;
wherein the presence of a reporter gene facilitates the ability of said yeast to grow in a leucine deficient media;
wherein a color change is generated by the cleavage of a substrate by the gene product,
wherein the intensity of said color is measured by colorimetry and quantified with OD units; and
wherein said OD units provide a read out value which may be further analysed to qualitatively or quantitatively determine caspase activity in the system.

17. The method of claim 16 wherein said reporter gene LEU 2 facilitates the ability of said yeast to grow in a leucine deficient media.

18. A method of generating and using a genetic system for monitoring from exogenous caspase activation pathways in yeast comprising:
a single-component system wherein said exogenous caspase is stably over-expressed and autoactivated in a yeast strain containing a transmembrane cleavable transcription factor;
wherein a chimera is expressed such that the majority of the cytosolic domain of said transmembrane receptor is replaced by a chimeric transcription factor;
wherein said chimeric transcription factor comprises the DNA-binding domain of LexA and the transactivation domain of B42 and is described as LexA-B42 chimeric transcription factor;
wherein between the Fas and the LexA-B42 domains are one or more tetrapeptide sequences known to be recognized and cleaved by various members of the Caspase family;
wherein an appropriately matched set of the appropriate caspase to the appropriate chimera results in a cleavable transcription factor wherein said transcription factor induces the expression of the LacZ and LEU2 genes;
wherein the presence of said LEU2 genes facilitate the ability of said yeast to grow in a leucine deficient media;
wherein a blue color culture is generated by the cleavage of X-gal by the LacZ gene product, β-galactosidase;
wherein the intensity of said blue color is measured by colorimetry and quantified with OD units; and
wherein said OD units provide a read out value which may be further analysed to qualitatively or quantitatively determine caspase activity in the system.

19. A method of using the single component system of claim 16 for drug screening assays where the activities of Caspase 1, 2, 3, 4, 5, 6, 7, 8, 9, and 10 are measured and compounds that inhibit Caspase 1, 2, 3, 4, 5, 6, 7, 8, 9, and 10 are identified.

20. A method of generating and using a genetic system for monitoring from exogenous caspase activation pathways in yeast comprising:
a two-component system comprising;
an exogenous caspase which is expressed at a low level to maintain an inactive pro-caspase form, wherein the level of expression of said exogenous caspase is engineered to prevent autoactivation;
an exogenous upstream or downstream activator;
wherein said transcription factor is expressed such that a the majority of the cytosolic domain of said transmembrane receptor is replaced by a chimeric transcription factor;
wherein said chimeric transcription factor comprises the DNA-binding domain of LexA and the transactivation domain of B42 and is described as LexA-B42 chimeric transcription factor;
wherein between the Fas and the LexA-B42 domains are one or more tetrapeptide sequences known to be recognized and cleaved by various members of the Caspase family;
wherein an appropriately matched set of the appropriate caspase to an appropriate chimera results in a cleavable transcription factor
wherein said transcription factor induces the expression of one or more reporter genes;
wherein the presence one or more receptor genes facilitate the ability of said yeast to grow in a leucine deficient media;
wherein a color culture comprises a color change generated by the cleavage of a substrate by the gene product;
wherein the intensity of the color is measured by colorimetry and quantified with OD units; and
wherein said OD units provide a read out value which may be further analysed to qualitatively or quantitatively determine caspase activity in the system.

21. A method of generating and using a genetic system for monitoring from exogenous caspase activation pathways in yeast comprising:
a two-component system comprising;
an exogenous caspase which is expressed at a low level to maintain an inactive pro-caspase form, wherein the level of expression of said exogenous caspase is engineered to prevent autoactivation;
an exogenous upstream or downstream activator;
wherein the combination of said exogenous caspase and said exogenous activator containing the transmembrane receptor, CD95/Fas, chimera;
wherein said chimera is expressed such that a the majority of the cytosolic domain of said transmembrane receptor is replaced by a chimeric transcription factor;
wherein said chimeric transcription factor comprises the DNA-binding domain of LexA and the transactivation domain of B42 and is described as LexA-B42 chimeric transcription factor;
wherein between the Fas and the LexA-B42 domains are a plurality of tetrapeptide sequences known to be recognized and cleaved by various members of the Caspase family;
wherein an appropriately matched set of the appropriate non-autoactivated exogenous caspase with an exogenous upstream or downstream activator results in the production of a transcription factor
wherein said transcription factor induces the expression of the LacZ and LEU2 genes;
wherein the presence of said LacZ and LEU2 genes facilitate the ability of said yeast to grow in a leucine deficient media;
wherein a blue color culture is generated by the cleavage of X-gal by the LacZ gene product, β-galactosidase;
wherein the intensity of said blue color is measured by colorimetry and quantified with OD units; and
wherein said OD units provide a read out value which may be further analysed to qualitatively or quantitatively determine caspase activity in the system.

22. A method of using the two component system of claim 21 for drug screening assays where the activities of ASC, RAIDD, FADD, Apaf, and active caspase-9 are measured and compounds that inhibit the interaction or activity of ASC with pro-caspase-1, RAIDD with pro-caspase-2, FADD with pro-caspase-8 or pro-caspase-10, an active mutant of Apaf with pro-caspase-9, active caspase-9 with pro-caspase-3 or procaspase-7 are identified.

23. The method of claim 22 where the activity of ASC with pro-caspase-1, RAIDD with pro-caspase-2, FADD with pro-caspase-8 or pro-caspase-10, and/or the activity of an active mutant of Apaf with pro-caspase-22 is identified.

24. The method of claim 22 where the activity of an active caspase-9 with pro-caspase-3 or procaspase-7 is identified.

25. A method of using a two-component system of claim 20 to identify known or novel upstream activators of pro-caspase-1, pro-caspase-2, pro-caspase-3, pro-caspase-4, pro-caspase-5, pro-caspase-6, pro-caspase-7, pro-caspase-8, pro-caspase-9, and pro-caspase-10;
via the introduction of a cDNA library or other expression conveyance to screen for biomolecular activators of the pro-caspases.

26. A method of generating and using a genetic system for monitoring from exogenous caspase activation pathways in yeast comprising:
a plural-component system comprising;
an exogenous caspase which is expressed at a low level to maintain an inactive pro-caspase form, wherein the level of expression of said exogenous caspase is engineered to prevent autoactivation;
two or more exogenous upstream or downstream activators; which individually are expressed at a low level to maintain an inactive form, wherein the level of expression of said exogenous activators are engineered to prevent caspase activation unless all three components are present;
wherein the combination of said exogenous caspase and said exogenous activator contains a transmembrane cleavable transcription factor;
wherein a chimera is expressed such that the majority of the cytosolic domain of said transmembrane receptor is replaced by a chimeric or transcription factor;
wherein said chimeric transcription factor comprises the DNA-binding domain of LexA and the transactivation domain of B42 and is described as LexA-B42 chimeric transcription factor;
wherein between the Fas and the LexA-B42 domains are a plurality of tetrapeptide sequences known to be recognized and cleaved by various members of the Caspase family;
wherein an appropriately matched set of the appropriate non-autoactivated exogenous caspase with the appropriate combination of two or more exogenous upstream or downstream activators results in the production of a transcription factor;
wherein said transcription factor induces the expression of the LacZ and LEU2 genes;
wherein the presence of said LacZ and LEU2 genes facilitate the ability of said yeast to grow in a leucine deficient media;
wherein a blue color culture is generated by the cleavage of X-gal by the LacZ gene product, β-galactosidase;
wherein the intensity of said blue color is measured by colorimetry and quantified with OD units; and
wherein said OD units provide a read out value which may be further analysed to qualitatively or quantitatively determine caspase activity in the system.

27. A method of using a plural component system of claim 26 for drug screening assays where the activities of FAS, FADD, DR5, ASC, or NALP1 are measured and compounds that inhibit the activity of FAS with FADD, DR5 with FADD, FADD with pro-caspase-8 or pro-caspase-10, ASC with NALP1 or other members of the NOD-like receptor family, or NALP1 or other members of the NOD-like receptor family with pro-caspase-1.

28. The method of claim 27 where the activity of FAS with FADD is identified.

29. The method of claim 27 where the activity of DR5 with FADD is identified.

30. The method of claim 27 where the activity of FADD with pro-caspase-8 or pro-caspase-10 is identified.

31. The method of claim 27 where the activity of ASC with NALP1 or other members of the NOD-like receptor family is identified.

32. The method of claim 27 where the activity of NALP1 with pro-caspase-1 is identified.

33. A method of using a plural component system of claim 16 to identify known or novel upstream activators of FADD and NALP1 via the introduction of a cDNA library or other expression conveyance to screen for biomolecular activators of FADD or NALP1.

34. A method of using a plural component system of claim 16 to identify known or novel downstream activators of FAS, DR5, or ASC via the introduction of a cDNA library or other expression conveyance to screen for biomolecular activators of FAS, DR5, or ASC.

35. A high throughput screening assay system for identifying lac-Z reporter gene activity comprising;
measuring β-galactosidase produced by yeast carrying caspase-cleavable reporter proteins;
and assaying the calorimetric product derived from x-gal substrate in 384 well plates, at an optical density of 620 nm.

36. A reporter gene system for monitoring protease activity in living yeast comprising:
singly or in concert a exogenous recombinant protease and/or a exogenous upstream activator of said protease;
wherein said recombinant protease and/or exogenous upstream activator cleave a chimeric protein;
wherein said chimeric protein facilitates a transcription factor;
wherein said transcription factor induces the expression of the LacZ and LEU2 genes; or any other substrate of the LacZ gene, beta-galactosidase, for quantification.

37. The reporter gene system of claim 36 wherein;
the presence of said LacZ and LEU2 genes facilitate the ability of said yeast to grow in a leucine deficient media;
a blue color culture is generated by the cleavage of X-gal by the LacZ gene product, β-galactosidase; and
said blue color culture assists with determining the protease activity of said exogenous recombinant protease and/or said exogenous upstream activator of said protease.

38. The reporter gene system of claim 36 wherein said protease activity is caspase activity; and wherein said exogenous recombinant protease is caspase;
wherein said exogenous recombinant protease is a exogenous recombinant caspase and said exogenous upstream activator of said protease is a exogenous upstream activator of caspase when present.

39. The reporter gene system of claim 38 wherein;
the intensity of said blue color is measured by colorimetry and quantified with OD units;
wherein said OD units are directly proportional to the activity of the caspase in the system;
wherein said OD units provide a read out value which may be further analysed to qualitatively or quantitatively determine caspase activity in the system.

40. A method of using a two-component and/or a plural component system to identify caspase activation protease networks in yeast, and to isolate and study an exogenous protease network in a cellular context.
